(19) United States
(12) Patent Application Publication
(10) Pub. No.: US 2021/0054389 A1
(43) Pub. Date: Feb. 25, 2021

(54) TRANSFORMATION OF DICOT PLANTS

(71) Applicant: PIONEER HI-BRED INTERNATIONAL, INC., JOHNSTON, IA (US)

(72) Inventors: HYEON-JE CHO, ANKENY, IA (US); TAI MINH, SAN JOSE, CA (US)

(73) Assignee: PIONEER HI-BRED INTERNATIONAL, INC., JOHNSTON, IA (US)

(21) Appl. No.: 16/957,869

(22) PCT Filed: Dec. 19, 2018
(86) PCT No.: PCT/US18/66460

§ 371 Date: Jun. 25, 2020

Related U.S. Application Data
(60) Provisional application No. 62/610,540, filed on Dec. 27, 2017.

(51) Int. Cl. C12N 15/82; C12N 9/22

(52) U.S. Cl. CPC C12N 15/8205; C12N 9/22; C12N 15/8207

(57) ABSTRACT

Compositions and methods are provided for the efficient transformation of a dicot plant. More particularly, compositions and methods of the present disclosure find use in agriculture for bacteria-mediated and biolistic-mediated transformation of a dicotyledonous plant. The compositions include cultivation media comprising methionine and glutamine supplemented with auxins for use in methods directed to bacteria-mediated and biolistic-mediated transformation of a dicot plant with a gene of interest.

### CROSS-REFERENCE TO RELATED APPLICATIONS

This application claims the benefit of PCT Application Serial Number PCT/US2018/066460, filed Dec. 19, 2018, which claims the benefit of U.S. Provisional Application No. 62/610,540, filed Dec. 27, 2017, both of which are hereby incorporated herein by reference in their entireties.

### FIELD OF THE DISCLOSURE

The present disclosure relates to the field of plant biotechnology. More particularly, the present disclosure relates to compositions and methods for bacteria-mediated and biolistic-mediated transformation of dicotyledonous plants.

### BACKGROUND

Cultivated dicotyledonous crops such as soybean, Brassica, and cotton have substantial commercial value throughout the world. The development of scientific methods useful in improving the quantity and quality of soybean and other crops is, therefore, of significant commercial interest. Significant effort has been expended to improve the quality of cultivated dicotyledonous crop species by conventional plant breeding. Methods of conventional plant breeding have been limited, however, to the movement of genes and traits between plant varieties.

In addition to traditional breeding techniques, incorporation of insect resistance, disease resistance, increased or modified oil content, and other desirable traits can be envisioned using the modern tools of molecular biology including plant genetic engineering. Plant genetic engineering involves the transfer of a desired gene or genes into the inheritable germline of crop plants such that those genes can be bred into or among the elite varieties used in modern agriculture. Gene transfer techniques allow the development of new classes of crop varieties with improved insect resistance, disease resistance, herbicide tolerance, and increased nutritional value.

Agrobacterium and biolistic methods have been widely used for the transformation of plants. Agrobacterium is a soil born phytopathogen that integrates a nucleic acid molecule (i.e., T-DNA) into the genome of a large number of dicotyledonous plants. Agrobacterium-mediated transformation involves incubation of cells or tissues with the bacterium, followed by regeneration of plants from the transformed cells via a callus stage. The advantage of Agrobacterium-mediated gene transfer is that it offers the potential to regenerate transgenic cells at relatively high frequencies without a significant reduction in plant regeneration rates. Moreover, the process of DNA transfer to the plant genome is defined. That is, the DNA does not normally undergo any major rearrangements, and it integrates into the genome often in single or low copy numbers. Biolistic transformation and the inoculation of a plant tissue with Agrobacterium is a disruptive process that can damage plant tissue and trigger a hypersensitive response in the tissue. As a result, the target tissue may become necrotic and the overall survival rate of transformants can be limited.

Accordingly, there remains a need for improved transformation methods to promote the engineering of desirable traits into agronomically important crops, such as soybean. In addition, there remains a need for highly efficient transformation methods that yield regenerable plant tissue.

### SUMMARY

Compositions and methods are provided for the transformation of plants. More particularly, compositions and methods of the present disclosure find use in agriculture for bacteria-mediated and biolistic-mediated transformation of a dicotyledonous plant. The compositions include cultivation media comprising concentrations of methionine and glutamine. The cultivation media find use in methods directed to bacteria-mediated and biolistic-mediated transformation of a dicot plant with a gene of interest. The cultivation media may also be used in a pre-cultivation/pre-incubation step prior to bacteria-mediated and biolistic-mediated transformation. The dicot plants include, but are not limited to, kale, cauliflower, broccoli, mustard plant, cabbage, pea, clover, alfalfa, broad bean, tomato, cassava, soybean, peanut, canola, alfalfa, sunflower, safflower, tobacco, Arabidopsis, and cotton. The dicot plant tissue includes, but is not limited to, immature cotyledon.

In an aspect, the present disclosure provides methods for transforming a dicot immature cotyledon to produce a fertile transgenic dicot plant, comprising (a) infecting and co-cultivating the immature cotyledon with bacteria containing a plasmid in M5 medium, (b) recovering transformed immature cotyledons in S30 medium, (c) selecting the transformed immature cotyledon in S30 medium comprising a selection agent, (d) obtaining transformed embryogenic callus from the immature cotyledon, and (e) regenerating transformed plants from the embryogenic callus. In an aspect, the immature cotyledon and the bacteria are co-cultivated on filter paper. In an aspect, the infecting is performed by wounding the immature cotyledon. In an aspect, the infecting is performed without wounding the immature cotyledon. In an aspect, the immature cotyledon is about 0.5-9.0 mm in length. In an aspect, the bacteria is a disarmed Agrobacteria, an Ochrobactrum bacteria or a Rhizobiaceae bacteria. In an aspect, the plasmid comprises a gene of interest and/or a selection marker. In an aspect, the plasmid comprises a guide RNA, a target nucleotide present in the dicot immature cotyledon, and a Cas endonuclease, wherein the guide RNA and the Cas endonuclease are capable of forming a complex that enables the Cas endonuclease to introduce a double strand break at a target site in the genome of the dicot immature cotyledon, wherein at least one nucleotide of the target nucleotide is modified. In an aspect, the plasmid comprises a promoter operably linked to a nucleotide sequence encoding an engineered meganuclease wherein the engineered meganuclease is capable of specifically binding to and creating a double strand break in a target sequence present in the dicot immature cotyledon and wherein the promoter is capable of driving expression of an operably linked nucleotide sequence in the dicot immature cotyledon, wherein the nucleotide sequence comprises an alteration in the genome of the dicot immature cotyledon. In an aspect, the selection agent is an antibiotic, an herbicide, or a positive selection agent. In an aspect, the selection agent is hygromycin, spectinomycin, kanamycin, a sulfonylurea herbicide, an imidazolinone herbicide, bialaphos, phosphinothricin, glyphosate, or a combination thereof. In an aspect, the sulfonylurea herbicide is chlorsulfuron, ethametsulfuron, or a combination thereof. In an aspect, the imidazolinone herbicide is imazapyr. In an aspect, the dicot plant is selected from the group consisting of kale, cauliflower, broccoli, mustard plant, cabbage, pea, clover, alfalfa, broad bean, tomato, cassava, soybean, peanut, canola, alfalfa, sunflower, safflower, tobacco, Arabidopsis, and cotton. In an aspect, the dicot plant is soybean.

In an aspect, the present disclosure provides methods for transforming a dicot immature cotyledon to produce a fertile transgenic dicot plant, comprising (a) infecting and co-cultivating the immature cotyledon with bacteria containing a plasmid in M5 medium, (b) recovering transformed immature cotyledons in Base medium supplemented with 2,4-D, (c) selecting the transformed immature cotyledon in Base medium comprising a selection agent, (d) obtaining transformed embryogenic callus from the immature cotyledon, and (e) regenerating transformed plants from the embryogenic callus. In an aspect, the 2,4-D is present in the Base medium in an amount from about 0.5 mg/liter to about 40 mg/liter. In a further aspect, the 2,4-D is present in the Base medium in an amount of about 10 mg/liter. In an aspect, the immature cotyledon and the bacteria are co-cultivated on filter paper. In an aspect, the infecting is performed by wounding the immature cotyledon. In an aspect, the infecting is performed without wounding the immature cotyledon. In an aspect, the immature cotyledon is about 0.5-9.0 mm in length. In an aspect, the bacteria is a disarmed Agrobacteria, an Ochrobactrum bacteria or a Rhizobiaceae bacteria. In an aspect, the plasmid comprises a gene of interest and/or a selection marker. In an aspect, the plasmid comprises a guide RNA, a target nucleotide present in the dicot immature cotyledon, and a Cas endonuclease, wherein the guide RNA and the Cas endonuclease are capable of forming a complex that enables the Cas endonuclease to introduce a double strand break at a target site in the genome of the dicot immature cotyledon, wherein at least one nucleotide of the target nucleotide is modified. In an aspect, the plasmid comprises a promoter operably linked to a nucleotide sequence encoding an engineered meganuclease wherein the engineered meganuclease is capable of specifically binding to and creating a double strand break in a target sequence present in the dicot immature cotyledon and wherein the promoter is capable of driving expression of an operably linked nucleotide sequence in the dicot immature cotyledon, wherein the nucleotide sequence comprises an alteration in the genome of the dicot immature cotyledon. In an aspect, the selection agent is an antibiotic, an herbicide, or a positive selection agent. In an aspect, the selection agent is hygromycin, spectinomycin, kanamycin, a sulfonylurea herbicide, an imidazolinone herbicide, bialaphos, phosphinothricin, glyphosate, or a combination thereof. In an aspect, the sulfonylurea herbicide is chlorsulfuron, ethametsulfuron, or a combination thereof. In an aspect, the imidazolinone herbicide is imazapyr. In an aspect, the dicot plant is selected from the group consisting of kale, cauliflower, broccoli, mustard plant, cabbage, pea, clover, alfalfa, broad bean, tomato, cassava, soybean, peanut, canola, alfalfa, sunflower, safflower, tobacco, Arabidopsis, and cotton. In an aspect, the dicot plant is soybean.

In an aspect, the present disclosure provides methods for transforming a dicot immature cotyledon to produce a fertile transgenic dicot plant, comprising (a) bombarding the immature cotyledon with a DNA or a ribonucleoprotein, (b) selecting the transformed immature cotyledon in S30 medium comprising a selection agent, (c) obtaining transformed embryogenic callus from the immature cotyledon, and (d) regenerating transformed plants from the embryogenic callus. In an aspect, the immature cotyledon is about 0.5-9.0 mm in length. In an aspect, the DNA or the ribonucleoprotein comprises a gene of interest and/or a selection marker. In an aspect, the DNA or the ribonucleoprotein comprises a guide RNA, a target nucleotide present in the dicot immature cotyledon, and a Cas endonuclease, wherein the guide RNA and the Cas endonuclease are capable of forming a complex that enables the Cas endonuclease to introduce a double strand break at a target site in the genome of the dicot immature cotyledon, wherein at least one nucleotide of the target nucleotide is modified. In an aspect, the DNA or the ribonucleoprotein comprises a promoter operably linked to a nucleotide sequence encoding an engineered meganuclease wherein the engineered meganuclease is capable of specifically binding to and creating a double strand break in a target sequence present in the dicot immature cotyledon and wherein the promoter is capable of driving expression of an operably linked nucleotide sequence in the dicot immature cotyledon, wherein the nucleotide sequence comprises an alteration in the genome of the dicot immature cotyledon. In an aspect, the selection agent is an antibiotic, an herbicide, or a positive selection agent. In an aspect, the selection agent is hygromycin, spectinomycin, kanamycin, a sulfonylurea herbicide, an imidazolinone herbicide, bialaphos, phosphinothricin, glyphosate, or a combination thereof. In an aspect, the sulfonylurea herbicide is chlorsulfuron, ethametsulfuron, or a combination thereof. In an aspect, the imidazolinone herbicide is imazapyr. In an aspect, the dicot plant is selected from the group consisting of kale, cauliflower, broccoli, mustard plant, cabbage, pea, clover, alfalfa, broad bean, tomato, cassava, soybean, peanut, canola, alfalfa, sunflower, safflower, tobacco, Arabidopsis, and cotton. In an aspect, the dicot plant is soybean.

In an aspect, the present disclosure provides methods for transforming a dicot immature cotyledon to produce a fertile transgenic dicot plant, comprising (a) bombarding the immature cotyledon with a DNA or a ribonucleoprotein, (b) selecting the transformed immature cotyledon in Base medium supplemented with 2,4-D and a selection agent, (c) obtaining transformed embryogenic callus from the immature cotyledon, and (d) regenerating transformed plants from the embryogenic callus. In an aspect, the 2,4-D is present in the Base medium in an amount from about 0.5 mg/liter to about 40 mg/liter. In a further aspect, the 2,4-D is present in the Base medium in an amount of about 10 mg/liter. In an aspect, the immature cotyledon is about 0.5-9.0 mm in length. In an aspect, the DNA or the ribonucleoprotein comprises a gene of interest and/or a selection marker. In an aspect, the DNA or the ribonucleoprotein comprises a guide RNA, a target nucleotide present in the dicot immature cotyledon, and a Cas endonuclease, wherein the guide RNA and the Cas endonuclease are capable of forming a complex that enables the Cas endonuclease to introduce a double strand break at a target site in the genome of the dicot immature cotyledon, wherein at least one nucleotide of the target nucleotide is modified. In an aspect, the DNA or the ribonucleoprotein comprises a promoter operably linked to a nucleotide sequence encoding an engineered meganuclease wherein the engineered meganuclease is capable of specifically binding to and creating a double strand break in a target sequence present in the dicot immature cotyledon and wherein the promoter is capable of driving expression of an operably linked nucleotide sequence in the dicot immature cotyledon, wherein the nucleotide sequence comprises an alteration in the genome of the dicot immature cotyledon. In an aspect, the selection agent is an antibiotic, an herbicide, or a positive selection agent. In an aspect, the selection agent is hygromycin, spectinomycin, kanamycin, a sulfonylurea herbicide, an imidazolinone herbicide, bialaphos, phosphinothricin, glyphosate, or a combination thereof. In an aspect, the sulfonylurea herbicide is chlorsulfuron, ethametsulfuron, or a combination thereof. In an aspect, the imidazolinone herbicide is imazapyr. In an aspect, the dicot plant is selected from the group consisting of kale, cauliflower, broccoli, mustard plant, cabbage, pea, clover, alfalfa, broad bean, tomato, cassava, soybean, peanut, canola, alfalfa, sunflower, safflower, tobacco, Arabidopsis, and cotton. In an aspect, the dicot plant is soybean.

In an aspect, the present disclosure provides methods for improving bacteria-mediated transformation frequency of a dicot immature cotyledon, comprising: culturing the immature cotyledon for a period of from about one day to about ten days in S30 medium prior to bacteria-mediated transformation to provide a pre-cultured immature cotyledon, wherein the bacteria-mediated transformation frequency of the pre-cultured immature cotyledon is improved when compared to the bacteria-mediated transformation frequency of a non-pre-cultured immature cotyledon.

In an aspect, the present disclosure provides methods for improving bacteria-mediated transformation frequency of a dicot immature cotyledon, comprising: culturing the immature cotyledon for a period of from about one day to about ten days in Base medium supplemented with 2,4-D prior to bacteria-mediated transformation to provide a pre-cultured immature cotyledon, wherein the bacteria-mediated transformation frequency of the pre-cultured immature cotyledon is improved when compared to the bacteria-mediated transformation frequency of a non-pre-cultured immature cotyledon. In an aspect, the 2,4-D is present in the Base medium in an amount from about 0.5 mg/liter to about 40 mg/liter. In a further aspect, the 2,4-D is present in the Base medium in an amount of about 10 mg/liter.

In an aspect, the present disclosure provides methods for improving biolistic-mediated transformation frequency of a dicot immature cotyledon, comprising: culturing the immature cotyledon for a period of from about one day to about ten days in S30 medium prior to biolistic-mediated transformation to provide a pre-cultured immature cotyledon, wherein the biolistic-mediated transformation frequency of the pre-cultured immature cotyledon is improved when compared to the biolistic-mediated transformation frequency of a non-pre-cultured immature cotyledon.

In an aspect, the present disclosure provides methods for transforming a dicot immature cotyledon to produce a fertile transgenic dicot plant, comprising (a) infecting and co-cultivating the immature cotyledon with bacteria containing a plasmid in M5 medium, (b) recovering transformed immature cotyledons in Modified S30 medium, (c) selecting the transformed immature cotyledon in Modified S30 medium comprising a selection agent, (d) obtaining transformed embryogenic callus from the immature cotyledon, and (e) regenerating transformed plants from the embryogenic callus. In an aspect, the immature cotyledon and the bacteria are co-cultivated on filter paper. In an aspect, the infecting is performed by wounding the immature cotyledon. In an aspect, the infecting is performed without wounding the immature cotyledon. In an aspect, the immature cotyledon is about 0.5-9.0 mm in length. In an aspect, the bacteria is a disarmed Agrobacteria, an Ochrobactrum bacteria or a Rhizobiaceae bacteria. In an aspect, the plasmid comprises a gene of interest and/or a selection marker. In an aspect, the plasmid comprises a guide RNA, a target nucleotide present in the dicot immature cotyledon, and a Cas endonuclease, wherein the guide RNA and the Cas endonuclease are capable of forming a complex that enables the Cas endonuclease to introduce a double strand break at a target site in the genome of the dicot immature cotyledon, wherein at least one nucleotide of the target nucleotide is modified. In an aspect, the plasmid comprises a promoter operably linked to a nucleotide sequence encoding an engineered meganuclease wherein the engineered meganuclease is capable of specifically binding to and creating a double strand break in a target sequence present in the dicot immature cotyledon and wherein the promoter is capable of driving expression of an operably linked nucleotide sequence in the dicot immature cotyledon, wherein the nucleotide sequence comprises an alteration in the genome of the dicot immature cotyledon. In an aspect, the selection agent is an antibiotic, an herbicide, or a positive selection agent. In an aspect, the selection agent is hygromycin, spectinomycin, kanamycin, a sulfonylurea herbicide, an imidazolinone herbicide, bialaphos, phosphinothricin, glyphosate, or a combination thereof. In an aspect, the sulfonylurea herbicide is chlorsulfuron, ethametsulfuron, or a combination thereof. In an aspect, the imidazolinone herbicide is imazapyr. In an aspect, the dicot plant is selected from the group consisting of kale, cauliflower, broccoli, mustard plant, cabbage, pea, clover, alfalfa, broad bean, tomato, cassava, soybean, peanut, canola, alfalfa, sunflower, safflower, tobacco, Arabidopsis, and cotton. In an aspect, the dicot plant is soybean.

In an aspect, the present disclosure provides methods for transforming a dicot immature cotyledon to produce a fertile transgenic dicot plant, comprising (a) infecting and co-cultivating the immature cotyledon with bacteria containing a plasmid in M5 medium, (b) recovering transformed immature cotyledons in Base medium supplemented with 2,4-D, (c) selecting the transformed immature cotyledon in Base medium comprising a selection agent, (d) obtaining transformed embryogenic callus from the immature cotyledon, and (e) regenerating transformed plants from the embryogenic callus. In an aspect, the 2,4-D is present in the Base medium in an amount from about 0.5 mg/liter to about 40 mg/liter. In a further aspect, the 2,4-D is present in the Base medium in an amount of about 10 mg/liter. In an aspect, the immature cotyledon and the bacteria are co-cultivated on filter paper. In an aspect, the infecting is performed by wounding the immature cotyledon. In an aspect, the infecting is performed without wounding the immature cotyledon. In an aspect, the immature cotyledon is about 0.5-9.0 mm in length. In an aspect, the bacteria is a disarmed Agrobacteria, an Ochrobactrum bacteria or a Rhizobiaceae bacteria. In an aspect, the plasmid comprises a gene of interest and/or a selection marker. In an aspect, the plasmid comprises a guide RNA, a target nucleotide present in the dicot immature cotyledon, and a Cas endonuclease, wherein the guide RNA and the Cas endonuclease are capable of forming a complex that enables the Cas endonuclease to introduce a double strand break at a target site in the genome of the dicot immature cotyledon, wherein at least one nucleotide of the target nucleotide is modified. In an aspect, the plasmid comprises a promoter operably linked to a nucleotide sequence encoding an engineered meganuclease wherein the engineered meganuclease is capable of specifically binding to and creating a double strand break in a target sequence present in the dicot immature cotyledon and wherein the promoter is capable of driving expression of an operably linked nucleotide sequence in the dicot immature cotyledon, wherein the nucleotide sequence comprises an alteration in the genome of the dicot immature cotyledon. In an aspect, the selection agent is an antibiotic, an herbicide, or a positive selection agent. In an aspect, the selection agent is hygromycin, spectinomycin, kanamycin, a sulfonylurea herbicide, an imidazolinone herbicide, bialaphos, phosphinothricin, glyphosate, or a combination thereof. In an aspect, the sulfonylurea herbicide is chlorsulfuron, ethametsulfuron, or a combination thereof. In an aspect, the imidazolinone herbicide is imazapyr. In an aspect, the dicot plant is selected from the group consisting of kale, cauliflower, broccoli, mustard plant, cabbage, pea, clover, alfalfa, broad bean, tomato, cassava, soybean, peanut, canola, alfalfa, sunflower, safflower, tobacco, Arabidopsis, and cotton. In an aspect, the dicot plant is soybean.

In an aspect, the present disclosure provides methods for transforming a dicot immature cotyledon to produce a fertile transgenic dicot plant, comprising (a) bombarding the immature cotyledon with a DNA or a ribonucleoprotein, (b) selecting the transformed immature cotyledon in Modified S30 medium comprising a selection agent, (c) obtaining transformed embryogenic callus from the immature cotyledon, and (d) regenerating transformed plants from the embryogenic callus. In an aspect, the immature cotyledon is about 0.5-9.0 mm in length. In an aspect, the DNA or the ribonucleoprotein comprises a gene of interest and/or a selection marker. In an aspect, the DNA or the ribonucleoprotein comprises a guide RNA, a target nucleotide present in the dicot immature cotyledon, and a Cas endonuclease, wherein the guide RNA and the Cas endonuclease are capable of forming a complex that enables the Cas endonuclease to introduce a double strand break at a target site in the genome of the dicot immature cotyledon, wherein at least one nucleotide of the target nucleotide is modified. In an aspect, the DNA or the ribonucleoprotein comprises a promoter operably linked to a nucleotide sequence encoding an engineered meganuclease wherein the engineered meganuclease is capable of specifically binding to and creating a double strand break in a target sequence present in the dicot immature cotyledon and wherein the promoter is capable of driving expression of an operably linked nucleotide sequence in the dicot immature cotyledon, wherein the nucleotide sequence comprises an alteration in the genome of the dicot immature cotyledon. In an aspect, the selection agent is an antibiotic, an herbicide, or a positive selection agent. In an aspect, the selection agent is hygromycin, spectinomycin, kanamycin, a sulfonylurea herbicide, an imidazolinone herbicide, bialaphos, phosphinothricin, glyphosate, or a combination thereof. In an aspect, the sulfonylurea herbicide is chlorsulfuron, ethametsulfuron, or a combination thereof. In an aspect, the imidazolinone herbicide is imazapyr. In an aspect, the dicot plant is selected from the group consisting of kale, cauliflower, broccoli, mustard plant, cabbage, pea, clover, alfalfa, broad bean, tomato, cassava, soybean, peanut, canola, alfalfa, sunflower, safflower, tobacco, Arabidopsis, and cotton. In an aspect, the dicot plant is soybean.

In an aspect, the present disclosure provides methods for transforming a dicot immature cotyledon to produce a fertile transgenic dicot plant, comprising (a) bombarding the immature cotyledon with a DNA or a ribonucleoprotein, (b) selecting the transformed immature cotyledon in Base medium supplemented with 2,4-D and a selection agent, (c) obtaining transformed embryogenic callus from the immature cotyledon, and (d) regenerating transformed plants from the embryogenic callus. In an aspect, the 2,4-D is present in the Base medium in an amount from about 0.5 mg/liter to about 40 mg/liter. In a further aspect, the 2,4-D is present in the Base medium in an amount of about 10 mg/liter. In an aspect, the immature cotyledon is about 0.5-9.0 mm in length. In an aspect, the DNA or the ribonucleoprotein comprises a gene of interest and/or a selection marker. In an aspect, the DNA or the ribonucleoprotein comprises a guide RNA, a target nucleotide present in the dicot immature cotyledon, and a Cas endonuclease, wherein the guide RNA and the Cas endonuclease are capable of forming a complex that enables the Cas endonuclease to introduce a double strand break at a target site in the genome of the dicot immature cotyledon, wherein at least one nucleotide of the target nucleotide is modified. In an aspect, the DNA or the ribonucleoprotein comprises a promoter operably linked to a nucleotide sequence encoding an engineered meganuclease wherein the engineered meganuclease is capable of specifically binding to and creating a double strand break in a target sequence present in the dicot immature cotyledon and wherein the promoter is capable of driving expression of an operably linked nucleotide sequence in the dicot immature cotyledon, wherein the nucleotide sequence comprises an alteration in the genome of the dicot immature cotyledon. In an aspect, the selection agent is an antibiotic, an herbicide, or a positive selection agent. In an aspect, the selection agent is hygromycin, spectinomycin, kanamycin, a sulfonylurea herbicide, an imidazolinone herbicide, bialaphos, phosphinothricin, glyphosate, or a combination thereof. In an aspect, the sulfonylurea herbicide is chlorsulfuron, ethametsulfuron, or a combination thereof. In an aspect, the imidazolinone herbicide is imazapyr. In an aspect, the dicot plant is selected from the group consisting of kale, cauliflower, broccoli, mustard plant, cabbage, pea, clover, alfalfa, broad bean, tomato, cassava, soybean, peanut, canola, alfalfa, sunflower, safflower, tobacco, Arabidopsis, and cotton. In an aspect, the dicot plant is soybean.

In an aspect, the present disclosure provides methods for improving bacteria-mediated transformation frequency of a dicot immature cotyledon, comprising: culturing the immature cotyledon for a period of from about one day to about ten days in Modified S30 medium prior to bacteria-mediated transformation to provide a pre-cultured immature cotyledon, wherein the bacteria-mediated transformation frequency of the pre-cultured immature cotyledon is improved when compared to the bacteria-mediated transformation frequency of a non-pre-cultured immature cotyledon.

In an aspect, the present disclosure provides methods for improving bacteria-mediated transformation frequency of a dicot immature cotyledon, comprising: culturing the immature cotyledon for a period of from about one day to about ten days in Base medium supplemented with 2,4-D prior to bacteria-mediated transformation to provide a pre-cultured immature cotyledon, wherein the bacteria-mediated transformation frequency of the pre-cultured immature cotyledon is improved when compared to the bacteria-mediated transformation frequency of a non-pre-cultured immature cotyledon. In an aspect, the 2,4-D is present in the Base medium in an amount from about 0.5 mg/liter to about 40 mg/liter. In a further aspect, the 2,4-D is present in the Base medium in an amount of about 10 mg/liter.

In an aspect, the present disclosure provides methods for improving biolistic-mediated transformation frequency of a dicot immature cotyledon, comprising: culturing the immature cotyledon for a period of from about one day to about ten days in Modified S30 medium prior to biolistic-mediated transformation to provide a pre-cultured immature cotyledon, wherein the biolistic-mediated transformation frequency of the pre-cultured immature cotyledon is improved when compared to the biolistic-mediated transformation frequency of a non-pre-cultured immature cotyledon.

In an aspect, the present disclosure provides methods for improving biolistic-mediated transformation frequency of a dicot immature cotyledon, comprising: culturing the immature cotyledon for a period of from about one day to about ten days in Base medium supplemented with 2,4-D prior to biolistic-mediated transformation to provide a pre-cultured immature cotyledon, wherein the biolistic-mediated transformation frequency of the pre-cultured immature cotyledon is improved when compared to the biolistic-mediated transformation frequency of a non-pre-cultured immature cotyledon. In an aspect, the 2,4-D is present in the Base medium in an amount from about 0.5 mg/liter to about 40 mg/liter. In a further aspect, the 2,4-D is present in the Base medium in an amount of about 10 mg/liter.

In an aspect, the present disclosure provides methods for generating embryogenic callus for biolistic-mediated transformation, comprising: culturing an immature cotyledon for a period of from about ten days to about five weeks in S30 medium to provide the embryogenic callus for biolistic-mediated transformation.

In an aspect, the present disclosure provides methods for generating embryogenic callus for biolistic-mediated transformation, comprising: culturing an immature cotyledon for a period of from about ten days to about five weeks in Modified S30 medium to provide the embryogenic callus for biolistic-mediated transformation.

In an aspect, the present disclosure provides methods for generating embryogenic callus for biolistic-mediated transformation, comprising: culturing an immature cotyledon for a period of from about ten days to about five weeks in Base medium supplemented with 2,4-D to provide the embryogenic callus for biolistic-mediated transformation. In an aspect, the 2,4-D is present in the Base medium in an amount from about 0.5 mg/liter to about 40 mg/liter. In a further aspect, the 2,4-D is present in the Base medium in an amount of about 10 mg/liter. In a further aspect, the 2,4-D is present in the Base medium in an amount of about 17 mg/liter.

### BRIEF DESCRIPTION OF THE DRAWINGS

FIG. 1 depicts the effect of glutamine and L-methionine (S30 medium) on embryogenic callus induction from immature cotyledon of soybean elite varieties, Elite A, Elite B, Elite C, and Elite D, after three (3) weeks of culture when compared to three (3) weeks of culture in medium lacking glutamine and L-methionine (M2 medium).

FIG. 2A-FIG. 2E depicts the increased induction of embryogenic callus formation of M2 medium supplemented with glutamine and/or L-methionine after three (3) weeks of culture on the medium formulation treatments. FIG. 2A depicts the induction of embryogenic callus formation of Treatment 1—M2 medium; FIG. 2B depicts the increased induction of embryogenic callus formation of Treatment 2—M2 medium supplemented with 1 mM L-methionine; FIG. 2C depicts the increased induction of embryogenic callus formation of Treatment 3—M2 medium supplemented with 1 mM glutamine and 1 mM L-methionine; FIG. 2D depicts the increased induction of embryogenic callus formation of Treatment 4—M2 medium supplemented with 30.6 mM glutamine and 1 mM L-methionine (S30 medium); and FIG. 2E depicts the increased induction of embryogenic callus formation of Treatment 5—M2 medium supplemented with 30.6 mM glutamine.

FIG. 3A-FIG. 3F depicts transformed embryogenic cultures of elite soybean variety 93Y21 after six (6) weeks of selection on S30 medium supplemented with 10 μg/L chlorsulfuron. Transformation efficiencies of immature cotyledons were significantly increased in the S30 medium treatment (FIG. 3C and FIG. 3D) and the S30 medium pre-culture/pre-incubation treatment (FIG. 3E and FIG. 3F) compared to the M2 medium treatment (FIG. 3A and FIG. 3B).

FIG. 4A and FIG. 4B depict hygromycin resistant calli showing fluorescence. FIG. 4A shows somatic embryos forming on the surface of a cotyledon explant (indicated by arrows). FIG. 4B shows somatic embryos with marker gene expression under a florescence microscope.

FIG. 5A and FIG. 5B depict chlorsulfuron resistant calli showing fluorescence. FIG. 5A shows somatic embryos forming on the surface of a cotyledon explant (indicated by arrows). FIG. 5B shows somatic embryos with marker gene expression under a florescence microscope.

### DETAILED DESCRIPTION

The disclosures herein will be described more fully hereinafter with reference to the accompanying drawings, in which some, but not all possible aspects are shown. Indeed, disclosures may be embodied in many different forms and should not be construed as limited to the aspects set forth herein; rather, these aspects are provided so that this disclosure will satisfy applicable legal requirements.

Many modifications and other aspects disclosed herein will come to mind to one skilled in the art to which the disclosed compositions and methods pertain having the benefit of the teachings presented in the following descriptions and the associated drawings. Therefore, it is to be understood that the disclosures are not to be limited to the specific aspects disclosed and that modifications and other aspects are intended to be included within the scope of the appended claims. Although specific terms are employed herein, they are used in a generic and descriptive sense only and not for purposes of limitation.

It is also to be understood that the terminology used herein is for the purpose of describing particular aspects only and is not intended to be limiting. As used in the specification and in the claims, the term “comprising” can include the aspect of “consisting of.” Unless defined otherwise, all technical and scientific terms used herein have the same meaning as commonly understood by one of ordinary skill in the art to which the disclosed compositions and methods belong. In this specification and in the claims which follow, reference will be made to a number of terms which shall be defined herein.

Provided in the disclosure are compositions and methods, for example bacteria-mediated and biolistic-mediated methods, for transforming dicotyledonous plants. Compositions include cultivation media comprising concentrations of methionine and glutamine with auxin. The cultivation media of the present disclosure find use in methods directed to bacteria-mediated and/or biolistic-mediated transformation of a dicot plant with a gene of interest. In this manner, any gene of interest can be introduced into a dicot plant with high transformation efficiency and reduced tissue necrosis. The transferred gene will be present in the transformed plant in low copy number. Transformed plants, plant cells, and seeds are also disclosed herein.

In the description that follows, a number of terms are used extensively. Units, prefixes, and symbols are denoted in their International System of Units (SI) accepted form. Numeric ranges recited within the specification are inclusive of the numbers defining the range and include each integer within the defined range. The following definitions are provided to facilitate understanding of the disclosure.

### Plants and Plant Material

The plant used in the disclosed methods can be from a dicot, including, but not limited to, carrot, celery, cucumber, eggplant, lettuce, melon, peanut, pepper, potato, pumpkin, radish, spinach, squash, tomato, watermelon, kale, cauliflower, broccoli, mustard plant, cabbage, pea, clover, alfalfa, broad bean, tomato, cassava, soybean, canola, alfalfa, sunflower, safflower, tobacco, Arabidopsis, or cotton. Alternatively, the plant used in the disclosed methods can be a monocot, including, but not limited to, barley, maize, millet, oats, rice, rye, Setaria sp., sorghum, sugarcane, switchgrass, triticale, turfgrass, or wheat.

An immature cotyledon is defined as a cotyledon obtained from a dicot seed 5 to 28 days after pollination. A cotyledon is an embryonic leaf in seed-bearing plants, one or more of which are the first leaves to appear from a germinating seed.

A somatic embryo is defined as a multicellular structure derived from a single somatic cell that progresses through developmental stages that are similar to the development of a zygotic embryo, including formation of globular and transition-stage embryos and formation of an embryo axis. Single somatic embryos derived from a zygotic embryo germinate to produce non-chimeric plants, which may originally derive from a single-cell.

An embryogenic callus is defined as a friable or non-friable mixture of undifferentiated or partially undifferentiated cells which gives rise to proliferating primary and secondary somatic embryos capable of regenerating into mature fertile plants.

Germination is a process in which embryos form a viable plant.

A transgenic plant is defined as a mature, fertile plant that contains a transgene.

Compositions provided herein include cultivation media having methionine and glutamine supplied with auxins. In some aspects, a cultivation medium (S30, Table 1) for bacteria-mediated and/or biolistic-mediated transformation contains, for example, at least about 0.448 gram/L to about 8.96 gram/L glutamine and at least about 1.49 mg/L to about 149 mg/L methionine.

Compositions provided herein may further comprise an auxin (2,4-Dichlorophenoxyacetic acid (2,4-D)). Particular examples include S30 medium and Base medium containing from about 0.5 milligrams/liter to about 40 milligrams/liter 2,4-D. Other auxins useful in the present disclosure include, but are not limited to, IAA, NAA, IBA, dicamba, and picloram.

A cultivation medium provided herein may further comprise any other appropriate constituents including, without limitation, antioxidants, vitamins (e.g., B5 vitamins), salts, sorbitol, mannitol, maltose, magnesium chloride, casein hydrosylate, activated charcoal, acetosyringone, and agar.

In another aspect, a method for transforming a dicotyledonous plant, plant tissue, or plant cell is provided. The methods provided herein rely upon the use of bacteria-mediated and/or biolistic-mediated gene transfer to produce regenerable plant cells having a nucleotide sequence of interest. Bacteria useful in the methods of the disclosure include, but are not limited to, a disarmed Agrobacteria, an Ochrobactrum bacteria or a Rhizobiaceae bacteria.

Agrobacterium-mediated gene transfer exploits the natural ability of Agrobacterium tumefaciens to transfer DNA into plant chromosomes. Agrobacterium is a plant pathogen that transfers a set of genes encoded in a region called T-DNA of the Ti plasmid into plant cells at wound sites. The typical result of gene transfer is a tumorous growth called a crown gall in which the T-DNA is stably integrated into a host chromosome. The ability to cause crown gall disease can be removed by deletion of the genes in the T-DNA without loss of DNA transfer and integration. The DNA to be transferred is attached to border sequences that define the end points of an integrated T-DNA. Disarmed Agrobacteria useful in the present methods include, but are not limited to, AGL-1, EHA105, GV3101, and LBA4404.

Ochrobactrum bacteria useful in the present methods include, but are not limited to, Ochrobactrum haywardense H1 NRRL Deposit B-67078, Ochrobactrum cytisi, Ochrobactrum daejeonense, Ochrobactrum lupine, Ochrobactrum oryzae, Ochrobactrum tritici LBNL124-A-10, HTG3-C-07, Ochrobactrum pecoris, Ochrobactrum ciceri, Ochrobactrum daejeonense, Ochrobactrum gallinifaecis, Ochrobactrum grignonense, Ochrobactrum guangzhouense, Ochrobactrum haematophilum, Ochrobactrum intermedium, Ochrobactrum lupini, Ochrobactrum oryzae, Ochrobactrum pituitosum, Ochrobactrum pseudintermedium, Ochrobactrum pseudogrignonense, Ochrobactrum rhizosphaerae, Ochrobactrum thiophenivorans, and Ochrobactrum tritici.

Rhizobiaceae bacteria useful in the present methods include, but are not limited to, Rhizobium lusitanum, Rhizobium rhizogenes, Agrobacterium rubi, Rhizobium multihospitium, Rhizobium tropici, Rhizobium miluonense, Rhizobium leguminosarum, Rhizobium leguminosarum bv. trifolii, Rhizobium leguminosarum bv. phaseoli, Rhizobium leguminosarum. bv. viciae, Rhizobium leguminosarum Madison, Rhizobium leguminosarum USDA2370, Rhizobium leguminosarum USDA2408, Rhizobium leguminosarum USDA2668, Rhizobium leguminosarum 2370G, Rhizobium leguminosarum 2370LBA, Rhizobium leguminosarum 2048G, Rhizobium leguminosarum 2048LBA, Rhizobium leguminosarum bv. phaseoli 2668G, Rhizobium leguminosarum bv. phaseoli 2668LBA, Rhizobium leguminosarum RL542C, Rhizobium etli USDA 9032, Rhizobium etli bv. phaseoli, Rhizobium endophyticum, Rhizobium tibeticum, Rhizobium etli, Rhizobium pisi, Rhizobium phaseoli, Rhizobium fabae, Rhizobium hainanense, Arthrobacter viscosus, Rhizobium alamii, Rhizobium mesosinicum, Rhizobium sullae, Rhizobium indigoferae, Rhizobium gallicum, Rhizobium yanglingense, Rhizobium mongolense, Rhizobium oryzae, Rhizobium loessense, Rhizobium tubonense, Rhizobium cellulosilyticum, Rhizobium soli, Neorhizobium galegae, Neorhizobium vignae, Neorhizobium huautlense, Neorhizobium alkalisoli, Aureimonas altamirensis, Aureimonas frigidaquae, Aureimonas ureilytica. Aurantimonas coralicida, Fulvimarina pelagi, Martelella mediterranea, Allorhizobium undicola, Allorhizobium vitis, Allorhizobium borbor, Beijerinckia fluminensis, Agrobacterium larrymoorei, Agrobacterium radiobacter, Rhizobium selenitireducens corrig. Rhizobium rosettiformans, Rhizobium daejeonense, Rhizobium aggregatum, Pararhizobium capsulatum, Pararhizobium giardinii, Ensifer mexicanus, Ensifer terangae, Ensifer saheli, Ensifer kostiensis, Ensifer kummerowiae, Ensifer fredii, Sinorhizobium americanum, Ensifer arboris, Ensifer garamanticus, Ensifer meliloti, Ensifer numidicus, Ensifer adhaerens, Sinorhizobium sp., Sinorhizobium meliloti SD630, Sinorhizobium meliloti USDA1002, Sinorhizobium fredii USDA205, Sinorhizobium fredii SF542G, Sinorhizobium fredii SF4404, and Sinorhizobium fredii SM542C.

As used herein, “plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores. The term “plant tissue” includes differentiated and undifferentiated tissues including, but not limited to, roots, stems, shoots, leaves, pollen, seeds, tumor tissue and various forms of cells and culture (e.g., single cells, cotyledons, immature cotyledons, protoplasts, embryos and callus tissue). The plant tissue may be in a plant or in a plant organ, tissue or cell culture. Plant tissue may also be a soybean immature cotyledon.

As used herein, the term “immature cotyledon transformed with a nucleotide sequence of interest” refers to plant cells which contain a gene of interest or modified nucleotide sequence, or is a plant or plant cell which is descended from a transformed or modified plant or cell and which comprises the gene of interest or modified nucleotide sequence. A “control” or “control plant” or “control plant cell” provides a reference point for measuring changes in phenotype of the subject plant or plant cell. A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

In some aspects, methods for producing regenerable plant cells having a nucleotide sequence of interest can include the steps of:

(a) contacting a tissue from a dicotyledonous plant with a bacteria comprising a vector which comprises the nucleotide sequence, where the nucleotide sequence comprises at least an expression cassette comprising a gene which confers resistance to a selection agent;

(b) co-cultivating the tissue with said bacteria in the presence of a cultivation medium provided herein;

(c) culturing the tissue of step (b) in a medium comprising an antibiotic capable of inhibiting the growth of the bacteria and without a selection agent;

(d) selecting regenerable cells, including embryogenic callus, comprising the nucleotide sequence in a medium comprising a selection agent; and

(e) regenerating transformed plants from the regenerable cells.

In the contacting step, plant tissue to be transformed can be contacted with a bacteria, including a disarmed Agrobacteria, an Ochrobactrum bacteria or a Rhizobiaceae bacteria.

As used herein, the term “tissue” is intended to include a plant tissue such as embryogenic callus, immature and mature embryo, cotyledons and immature cotyledons, immature and mature seed, meristem, cell clusters, scutella, nodes, young leaf bases, hypocotyl explants, roots, embryonic axis, inflorescences, suspension cultures, cultures of suspended cell aggregates, meristematic regions, leaves, green tissue, non-green tissue, somatic embryos and shoot apexes and the like. As known to one skilled in the art, tissue may be obtained from any number of sources. For example, embryos can be obtained from the fertilized reproductive organs of a mature dicotyledonous plant. Embryogenic suspension cultures can be used for transformation. See, for example, US Patent Publication 2018/0216123; Finer and Naganawa, (1998) Plant Cell Tissue Org. Cult. 15:125-136; and Samoylov, et al., (1998) In Vitro Cellular and Developmental Biology—Plant 34:8-13, all of which are herein incorporated by reference in their entireties.

A variety of Agrobacterium species are known in the art, particularly for dicotyledon transformation. Such Agrobacterium can be used in the methods of the disclosure. See, for example, Hooykaas (1989) Plant Mol. Biol. 13:327; Smith, et al., (1995) Crop Science 35:301; Chilton, (1993) Proc. Natl. Acad. Sci. USA 90:3119; Mollony, et al., Monograph Theor Appl Genet NY, Springer Verlag 19:148, 1993 and Ishida, et al., (1996) Nature Biotechnol. 14:745; Komari, et al. (1996) The Plant Journal 10:165, herein incorporated by reference. See, also, DNA Cloning Service on the worldwide web at DNA-cloning.com.

Any disarmed Agrobacterium including, but not limited to AGL1, LBA4404, EHA105 and GV3101, any Ochrobactrum strain, or any Rhizobiaceae strain capable of delivering a gene of interest to a plant cell may be used in the methods of the disclosure. The bacteria strains utilized in the methods of the disclosure are modified to contain a gene or genes of interest, or a nucleic acid to be expressed in the transformed cells. The nucleic acid to be transferred is incorporated into the T-region and is flanked by T-DNA border sequences. In the Ti plasmid, the T-region is distinct from the vir region whose functions are responsible for transfer and integration. Binary vector systems have been developed where the manipulated disarmed T-DNA carrying foreign DNA and the vir functions are present on separate plasmids. In this manner, a modified T-DNA region comprising foreign DNA (the nucleic acid to be transferred) is constructed in a small plasmid which replicates in E. coli. This plasmid is transferred conjugatively in a tri-parental mating into A. tumefaciens which contains a compatible plasmid-carrying virulence gene. The vir functions are supplied in trans to transfer the T-DNA into the plant genome. Such binary vectors are useful in the practice of the present disclosure.

A vector comprising the nucleic acid of interest is introduced into an Agrobacterium, Ochrobactrum bacteria or a Rhizobiaceae bacteria. The term “introduced” is intended to mean providing a nucleic acid (e.g., expression construct) or protein into a cell (e.g., Agrobacterium or Ochrobactrum or Rhizobiaceae). “Introduced” includes reference to the incorporation of a nucleic acid into a eukaryotic or prokaryotic cell where the nucleic acid may be incorporated into the genome of the cell, and includes reference to the transient provision of a nucleic acid or protein to the cell. The term “introduced” includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid, or mitochondrial DNA), converted into an autonomous replicon, or transiently expressed (e.g., transfected mRNA). General molecular techniques used in the disclosure are provided, for example, by Sambrook, et al., (eds.) Molecular Cloning: A Laboratory Manual, 1989, Cold Spring Harbor Laboratory Press, Cold Spring Harbor, N.Y.

A DNA or a ribonucleoprotein can be introduced into a plant cell, including, but not limited to, a soybean immature cotyledon by microprojectile bombardment or particle gun bombardment-mediated transformation (i.e., biolistic-mediated transformation) as described in U.S. Pat. Nos. 5,015,580; 5,550,318; 5,538,880; 6,160,208; 6,399,861; and 6,403,865 all of which are incorporated herein by reference in their entirety.

For convenience, the nucleic acid to be transferred can be contained within DNA constructs or expression cassettes. The expression cassette or construct will comprise a transcriptional initiation region linked to the nucleic acid or gene of interest. Such an expression cassette is provided with a plurality of restriction sites for insertion of the gene or genes of interest to be under the transcriptional regulation of the regulatory regions. The expression cassettes may contain one or more than one gene or nucleic acid sequence to be transferred and expressed in the transformed plant. Thus, each nucleic acid sequence will be operably linked to 5′ and 3′ regulatory sequences. One or multiple expression cassettes or DNA constructs can be used in the practice of the disclosure.

The transcriptional initiation region, the promoter, may be native or homologous or foreign or heterologous to the host, or could be the natural sequence or a synthetic sequence. By foreign is intended that the transcriptional initiation region is not found in the wild-type host into which the transcriptional initiation region is introduced. As used herein a chimeric gene comprises a coding sequence operably linked to transcription initiation region which is heterologous to the coding sequence.

The transcriptional cassette will include in 5′-3′ direction of transcription, a transcriptional and translational initiation region, a DNA sequence of interest, and a transcriptional and translational termination region functional in plants. The termination region may be native with the transcriptional initiation region, may be native with the DNA sequence of interest, or may be derived from another source. Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., 1989) Nucleic Acids Res. 17:7891-7903; Joshi, et al., (1987) Nucleic Acid Res. 15:9627-9639.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein, et al., (1989) PNAS USA, 86:6126-6130); potyvirus leaders, for example, TEV leader (Tobacco Etch Virus) (Allison, et al., (1986); MDMV leader (Maize Dwarf Mosaic Virus); Virology, 154:9-20) and human immunoglobulin heavy-chain binding protein (BiP), (Macejak and Sarnow, (1991) Nature, 353:90-94; untranslated leader from the coat protein mRNA of alfalfa mosaic virus (AMV RNA 4), (Jobling and Gehrke, (1987) Nature 325:622-625; tobacco mosaic virus leader (TMV), (Gallie, et al., (1989) Molecular Biology of RNA, 237-256 and maize chlorotic mottle virus leader (MCMV) (Lommel, et al., (1991) Virology 81:382-385). See also, Della-Cioppa, et al., (1987) Plant Physiology, 84:965-968. Other methods known to enhance translation can also be utilized, for example, introns, and the like.

Generally, the expression cassette will comprise a selectable marker gene for the selection of transformed cells. Selectable marker genes are utilized for the selection of transformed cells or tissues. Selectable marker genes include genes encoding antibiotic resistance, such as those encoding neomycin phosphotransferase II (NEO) and hygromycin phosphotransferase (HPT) as well as genes conferring resistance to herbicidal compounds. Herbicide resistance genes generally code for a modified target protein insensitive to the herbicide or for an enzyme that degrades or detoxifies the herbicide in the plant before it can act. (See, DeBlock, et al., (1987) EMBO J. 6:2513-2518; DeBlock, et al., (1989) Plant Physiol. 91:691-704; Fromm, et al., (1990) 8:833-839; Gordon-Kamm, et al., (1990) 2:603-618). For example, resistance to glyphosate or sulfonylurea herbicides has been obtained by using genes coding for the mutant target enzymes, 5-enolpyruvylshikimate-3-phosphate synthase (EPSPS) and acetolactate synthase (ALS). Resistance to glufosinate ammonium, bromoxynil, and 2,4-dichlorophenoxyacetate (2,4-D) have been obtained by using bacterial genes encoding phosphinothricin acetyltransferase, a nitrilase, or a 2,4-dichlorophenoxyacetate monooxygenase, which detoxify the respective herbicides. Many selectable markers are known in the art and can be used in the practice of the disclosure.

Where appropriate, the selectable marker genes and other gene(s) and nucleic acid of interest to be transferred can be synthesized for optimal expression in a dicot (e.g., soybean). That is, the coding sequence of the genes can be modified to enhance expression in a dicot plant (e.g., soybean). The synthetic nucleic acid is designed to be expressed in the transformed tissues and plants at a higher level. The use of optimized selectable marker genes may result in higher transformation efficiency.

In an aspect, the disclosed methods and compositions can be used to introduce into plants with increased efficiency and speed polynucleotides useful to target a specific site for modification in the genome of a plant. Site specific modifications that can be introduced with the disclosed methods and compositions include those produced using any method for introducing site specific modification, including, but not limited to, through the use of gene repair oligonucleotides (e.g. US Publication 2013/0019349), or through the use of double-stranded break technologies such as TALENs, meganucleases, zinc finger nucleases, CRISPR-Cas, and the like.

In an aspect, the present disclosure comprises transformation methods and compositions for producing a transgenic dicot plant, wherein the method comprises (a) infecting and co-cultivating a dicot immature cotyledon in M5 medium with a bacteria containing a plasmid, (b) recovering transformed immature cotyledons in S30 medium without a selection agent, (c) selecting the transformed immature cotyledon in S30 medium with a selection agent, (d) obtaining transformed embryogenic callus from the immature cotyledon, and (e) regenerating transformed plants from the embryogenic callus, and wherein the plasmid further comprises a promoter operably linked to a nucleotide sequence encoding an engineered meganuclease wherein the engineered meganuclease is capable of specifically binding to and creating a double strand break in a target sequence present in the dicot immature cotyledon and wherein the promoter is capable of driving expression of an operably linked nucleotide sequence in the dicot immature cotyledon, wherein the nucleotide sequence comprises an alteration in the genome of the dicot immature cotyledon.

In an aspect, the present disclosure comprises transformation methods and compositions for producing a transgenic dicot plant, wherein the method comprises (a) bombarding a dicot immature cotyledon with DNA or ribonucleoprotein, (b) selecting the transformed immature cotyledon in S30 medium comprising a selection agent, (c) obtaining transformed embryogenic callus from the immature cotyledon, and (d) regenerating transformed plants from the embryogenic callus, wherein the DNA or the ribonucleoprotein further comprises a promoter operably linked to a nucleotide sequence encoding an engineered meganuclease wherein the engineered meganuclease is capable of specifically binding to and creating a double strand break in a target sequence present in the dicot immature cotyledon and wherein the promoter is capable of driving expression of an operably linked nucleotide sequence in the dicot immature cotyledon, wherein the nucleotide sequence comprises an alteration in the genome of the dicot immature cotyledon.

For example, the disclosed methods and compositions can be used to introduce a CRISPR-Cas system into plants, for the purpose of genome modification of a target sequence in the genome of a plant or plant cell, for selecting plants, for deleting a base or a sequence, for gene editing, and for inserting a polynucleotide of interest into the genome of a plant. Thus, the disclosed methods and compositions can be used together with a CRISPR-Cas system to provide for an effective system for modifying or altering target sites and nucleotides of interest within the genome of a plant, plant cell or seed.

In an aspect, the present disclosure comprises transformation methods and compositions for producing a transgenic dicot plant, wherein the method comprises (a) infecting and co-cultivating a dicot immature cotyledon in M5 medium with a bacteria containing a plasmid, (b) recovering transformed immature cotyledons in S30 medium without a selection agent, (c) selecting the transformed immature cotyledon in S30 medium with a selection agent, (d) obtaining transformed embryogenic callus from the immature cotyledon, and (e) regenerating transformed plants from the embryogenic callus, and wherein the plasmid further comprises a guide RNA, a target nucleotide present in the dicot immature cotyledon, and a Cas endonuclease, wherein the guide RNA and the Cas endonuclease are capable of forming a complex that enables the Cas endonuclease to introduce a double strand break at a target site in the genome of the dicot immature cotyledon, wherein at least one nucleotide of the target nucleotide is modified.

In an aspect, the present disclosure comprises transformation methods and compositions for producing a transgenic dicot plant, wherein the method comprises (a) bombarding a dicot immature cotyledon with DNA or ribonucleoprotein, (b) selecting the transformed immature cotyledon in S30 medium comprising a selection agent, (c) obtaining transformed embryogenic callus from the immature cotyledon, and (d) regenerating transformed plants from the embryogenic callus, wherein the DNA or the ribonucleoprotein further comprises a guide RNA, a target nucleotide present in the dicot immature cotyledon, and a Cas endonuclease, wherein the guide RNA and the Cas endonuclease are capable of forming a complex that enables the Cas endonuclease to introduce a double strand break at a target site in the genome of the dicot immature cotyledon, wherein at least one nucleotide of the target nucleotide is modified.

In an aspect, the Cas endonuclease gene is a plant optimized Cas9 endonuclease, wherein the plant optimized Cas9 endonuclease is capable of binding to and creating a double strand break in a genomic target sequence of the plant genome.

The Cas endonuclease is guided by the guide nucleotide to recognize and optionally introduce a double strand break at a specific target site into the genome of a cell. The CRISPR-Cas system provides for an effective system for modifying target sites within the genome of a plant, plant cell or seed. Further provided are methods and compositions employing a guide polynucleotide/Cas endonuclease system to provide an effective system for modifying target sites within the genome of a cell and for editing a nucleotide sequence in the genome of a cell. Once a genomic target site is identified, a variety of methods can be employed to further modify the target sites such that they contain a variety of polynucleotides of interest. The disclosed compositions and methods can be used to introduce a CRISPR-Cas system for editing a nucleotide sequence in the genome of a cell. The nucleotide sequence to be edited (the nucleotide sequence of interest) can be located within or outside a target site that is recognized by a Cas endonuclease.

CRISPR loci (Clustered Regularly Interspaced Short Palindromic Repeats) (also known as SPIDRs-SPacer Interspersed Direct Repeats) constitute a family of recently described DNA loci. CRISPR loci consist of short and highly conserved DNA repeats (typically 24 to 40 bp, repeated from 1 to 140 times, also referred to as CRISPR-repeats) which are partially palindromic. The repeated sequences (usually specific to a species) are interspaced by variable sequences of constant length (typically 20 to 58 by depending on the CRISPR locus (WO2007/025097 published Mar. 1, 2007).

CRISPR loci were first recognized in E. coli (Ishino et al. (1987) J. Bacterial. 169:5429-5433; Nakata et al. (1989) J. Bacterial. 171:3553-3556). Similar interspersed short sequence repeats have been identified in Haloferax mediterranei, Streptococcus pyogenes, Anabaena, and Mycobacterium tuberculosis (Groenen et al. (1993) Mol. Microbiol. 10:1057-1065; Hoe et al. (1999) Emerg. Infect. Dis. 5:254-263; Masepohl et al. (1996) Biochim. Biophys. Acta 1307:26-30; Mojica et al. (1995) Mol. Microbiol. 17:85-93). The CRISPR loci differ from other SSRs by the structure of the repeats, which have been termed short regularly spaced repeats (SRSRs) (Janssen et al. (2002) OMICS J. Integ. Biol. 6:23-33; Mojica et al. (2000) Mol. Microbiol. 36:244-246). The repeats are short elements that occur in clusters, that are always regularly spaced by variable sequences of constant length (Mojica et al. (2000) Mol. Microbiol. 36:244-246).

The Cas gene includes a gene that is generally coupled, associated or close to or in the vicinity of a flanking CRISPR loci. The terms “Cas gene” and “CRISPR-associated (Cas) gene” are used interchangeably herein. A comprehensive review of the Cas protein family is presented in Haft et al. (2005) Computational Biology, PLoS Comput Biol 1 (6): e60. doi:10.1371/journal.pcbi.0010060.

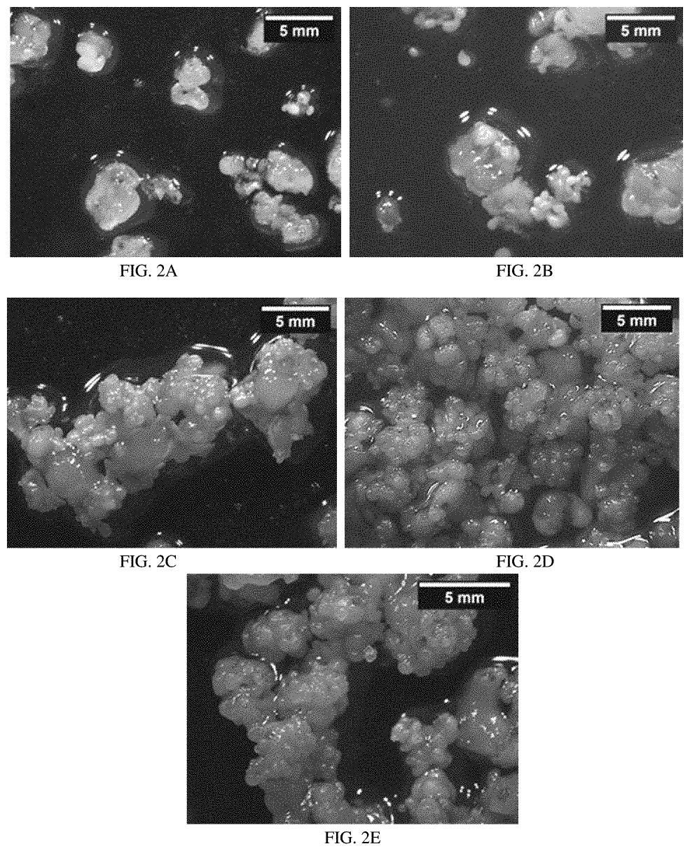

In addition to the four initially described gene families, an additional 41 CRISPR-associated (Cas) gene families have been described in WO/2015/026883, which is incorporated herein by reference. WO/2015/026883 shows that CRISPR systems belong to different classes, with different repeat patterns, sets of genes, and species ranges. The number of Cas genes at a given CRISPR locus can vary between species. Cas endonuclease relates to a Cas protein encoded by a Cas gene, wherein the Cas protein is capable of introducing a double strand break into a DNA target sequence. The Cas endonuclease is guided by the guide polynucleotide to recognize and optionally introduce a double strand break at a specific target site into the genome of a cell. As used herein, the term “guide polynucleotide/Cas endonuclease system” includes a complex of a Cas endonuclease and a guide polynucleotide that is capable of introducing a double strand break into a DNA target sequence. The Cas endonuclease unwinds the DNA duplex in close proximity of the genomic target site and cleaves both DNA strands upon recognition of a target sequence by a guide nucleotide, but only if the correct protospacer-adjacent motif (PAM) is approximately oriented at the 3′ end of the target sequence (see FIG. 2A and FIG. 2B of WO/2015/026883, published Feb. 26, 2015).

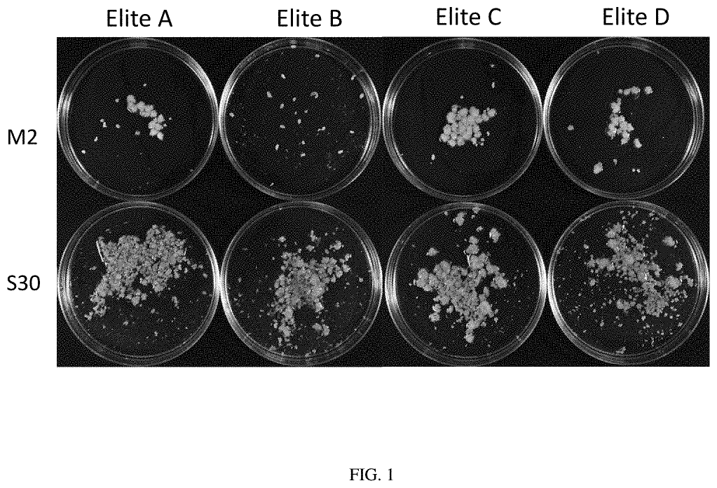

In an aspect, the Cas endonuclease gene is a Cas9 endonuclease, such as, but not limited to, Cas9 genes listed in SEQ ID NOs: 462, 474, 489, 494, 499, 505, and 518 of WO2007/025097, published Mar. 1, 2007, and incorporated herein by reference. In another aspect, the Cas endonuclease gene is a plant, maize or soybean optimized Cas9 endonuclease, such as, but not limited to those shown in FIG. 1A of WO/2015/026883. In another aspect, the Cas endonuclease gene is operably linked to a SV40 nuclear targeting signal upstream of the Cas codon region and a bipartite VirD2 nuclear localization signal (Tinland et al. (1992) Proc. Natl. Acad. Sci. USA 89:7442-6) downstream of the Cas codon region.

In an aspect, the Cas endonuclease gene is a Cas9 endonuclease gene of SEQ ID NO:1, 124, 212, 213, 214, 215, 216, 193 or nucleotides 2037-6329 of SEQ ID NO:5, or any functional fragment or variant thereof, of WO/2015/026883.

The terms “functional fragment,” “fragment that is functionally equivalent,” and “functionally equivalent fragment” are used interchangeably herein. These terms refer to a portion or subsequence of the Cas endonuclease sequence in which the ability to create a double-strand break is retained.

The terms “functional variant,” “variant that is functionally equivalent” and “functionally equivalent variant” are used interchangeably herein. These terms refer to a variant of the Cas endonuclease of the present disclosure in which the ability to create a double-strand break is retained. Fragments and variants can be obtained via methods such as site-directed mutagenesis and synthetic construction.

In an aspect, the Cas endonuclease gene is a plant codon optimized Streptococcus pyogenes Cas9 gene that can recognize any genomic sequence of the form N(12-30)NGG can in principle be targeted.

Endonucleases are enzymes that cleave the phosphodiester bond within a polynucleotide chain, and include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex.

Endonucleases also include meganucleases, also known as homing endonucleases (HEases), which like restriction endonucleases, bind and cut at a specific recognition site, however the recognition sites for meganucleases are typically longer, about 18 bp or more (Patent application PCT/US 12/30061 filed on Mar. 22, 2012). Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. Meganucleases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. This cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families. TAL effector nucleases are a new class of sequence-specific nucleases that can be used to make double-strand breaks at specific target sequences in the genome of a plant or other organism. (Miller, et al. (2011) Nature Biotechnology 29:143-148). Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a nonspecific endonuclease domain, for example nuclease domain from a Type Ms endonuclease such as Fokl. Additional functionalities can be fused to the zinc finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18-nucleotide recognition sequence.

Bacteria and archaea have evolved adaptive immune defenses termed clustered regularly interspaced short palindromic repeats (CRISPR)/CRISPR-associated (Cas) systems that use short RNA to direct degradation of foreign nucleic acids ((WO2007/025097 published Mar. 1, 2007). The type II CRISPR/Cas system from bacteria employs a crRNA and tracrRNA to guide the Cas endonuclease to its DNA target. The crRNA (CRISPR RNA) contains the region complementary to one strand of the double strand DNA target and base pairs with the tracrRNA (trans-activating CRISPR RNA) forming a RNA duplex that directs the Cas endonuclease to cleave the DNA target.

As used herein, the term “guide nucleotide” relates to a synthetic fusion of two RNA molecules, a crRNA (CRISPR RNA) comprising a variable targeting domain, and a tracrRNA. In an aspect, the guide nucleotide comprises a variable targeting domain of 12 to 30 nucleotide sequences and a RNA fragment that can interact with a Cas endonuclease.

As used herein, the term “guide polynucleotide” relates to a polynucleotide sequence that can form a complex with a Cas endonuclease and enables the Cas endonuclease to recognize and optionally cleave a DNA target site. The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide nucleotide”.

The guide polynucleotide can be a double molecule (also referred to as duplex guide polynucleotide) comprising a first nucleotide sequence domain (referred to as Variable Targeting domain or VT domain) that is complementary to a nucleotide sequence in a target DNA and a second nucleotide sequence domain (referred to as Cas endonuclease recognition domain or CER domain) that interacts with a Cas endonuclease polypeptide. The CER domain of the double molecule guide polynucleotide comprises two separate molecules that are hybridized along a region of complementarity. The two separate molecules can be RNA, DNA, and/or RNA-DNA-combination sequences. In an aspect, the first molecule of the duplex guide polynucleotide comprising a VT domain linked to a CER domain is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the cRNA naturally occurring in Bacteria and Archaea. In an aspect, the size of the fragment of the cRNA naturally occurring in Bacteria and Archaea that is present in a crNucleotide can range from, but is not limited to, 2, 3, 4, 5, 6, 7, 8, 9, 10, 11, 12, 13, 14, 15, 16, 17, 18, 19, 20 or more nucleotides.

In an aspect, the second molecule of the duplex guide polynucleotide comprising a CER domain is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides. In an aspect, the RNA that guides the RNA Cas9 endonuclease complex, is a duplexed RNA comprising a duplex crRNA-tracrRNA.

The guide polynucleotide can also be a single molecule comprising a first nucleotide sequence domain (referred to as Variable Targeting domain or VT domain) that is complementary to a nucleotide sequence in a target DNA and a second nucleotide domain (referred to as Cas endonuclease recognition domain or CER domain) that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. In an aspect, the single guide polynucleotide comprises a crNucleotide (comprising a VT domain linked to a CER domain) linked to a tracrNucleotide (comprising a CER domain), wherein the linkage is a nucleotide sequence comprising a RNA sequence, a DNA sequence, or a RNA-DNA combination sequence. The single guide polynucleotide being comprised of sequences from the crNucleotide and tracrNucleotide may be referred to as “single guide nucleotide” (when composed of a contiguous stretch of RNA nucleotides) or “single guide DNA” (when composed of a contiguous stretch of DNA nucleotides) or “single guide nucleotide-DNA” (when composed of a combination of RNA and DNA nucleotides). In an aspect of the disclosure, the single guide nucleotide comprises a cRNA or cRNA fragment and a tracrRNA or tracrRNA fragment of the type II CRISPR/Cas system that can form a complex with a type II Cas endonuclease, wherein the guide nucleotide Cas endonuclease complex can direct the Cas endonuclease to a plant genomic target site, enabling the Cas endonuclease to introduce a double strand break into the genomic target site. One aspect of using a single guide polynucleotide versus a duplex guide polynucleotide is that only one expression cassette needs to be made to express the single guide polynucleotide.

The term “variable targeting domain” or “VT domain” is used interchangeably herein and includes a nucleotide sequence that is complementary to one strand (nucleotide sequence) of a double strand DNA target site. The % complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable target domain can be at least 12, 13, 14, 15, 16, 17, 18, 19, 20, 21, 22, 23, 24, 25, 26, 27, 28, 29 or 30 nucleotides in length. In an aspect, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” of a guide polynucleotide is used interchangeably herein and includes a nucleotide sequence (such as a second nucleotide sequence domain of a guide polynucleotide), that interacts with a Cas endonuclease polypeptide. The CER domain can be composed of a DNA sequence, a RNA sequence, a modified DNA sequence, a modified RNA sequence (see for example modifications described herein), or any combination thereof.

The nucleotide sequence linking the crNucleotide and the tracrNucleotide of a single guide polynucleotide can comprise a RNA sequence, a DNA sequence, or a RNA-DNA combination sequence. In an aspect, the nucleotide sequence linking the crNucleotide and the tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another aspect, the nucleotide sequence linking the crNucleotide and the tracrNucleotide of a single guide polynucleotide can comprise a tetraloop sequence, such as, but not limiting to a GAAA tetraloop sequence.

Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

In an aspect, the guide nucleotide and Cas endonuclease are capable of forming a complex that enables the Cas endonuclease to introduce a double strand break at a DNA target site.

In an aspect of the disclosure the variable target domain is 12, 13, 14, 15, 16, 17, 18, 19, 20, 21, 22, 23, 24, 25, 26, 27, 28, 29 or 30 nucleotides in length.

In an aspect of the disclosure, the guide nucleotide comprises a cRNA (or cRNA fragment) and a tracrRNA (or tracrRNA fragment) of the type II CRISPR/Cas system that can form a complex with a type II Cas endonuclease, wherein the guide nucleotide Cas endonuclease complex can direct the Cas endonuclease to a plant genomic target site, enabling the Cas endonuclease to introduce a double strand break into the genomic target site. In an aspect, the guide nucleotide can be introduced into a plant or plant cell directly using any method known in the art such as, but not limited to, particle bombardment or topical applications.

In an aspect, the guide nucleotide can be introduced indirectly by introducing a recombinant DNA molecule comprising the corresponding guide DNA sequence operably linked to a plant specific promoter that is capable of transcribing the guide nucleotide in the plant cell. The term “corresponding guide DNA” includes a DNA molecule that is identical to the RNA molecule but has a “T” substituted for each “U” of the RNA molecule.

In an aspect, the guide nucleotide is introduced via particle bombardment or using the disclosed methods and compositions for Agrobacterium transformation of a recombinant DNA construct comprising the corresponding guide DNA operably linked to a plant U6 polymerase III promoter.

In an aspect, the RNA that guides the RNA Cas9 endonuclease complex, is a duplexed RNA comprising a duplex crRNA-tracrRNA. One advantage of using a guide nucleotide versus a duplexed crRNA-tracrRNA is that only one expression cassette needs to be made to express the fused guide nucleotide.

The terms “target site,” “target sequence,” “target DNA,” “target locus,” “genomic target site,” “genomic target sequence,” and “genomic target locus” are used interchangeably herein and refer to a polynucleotide sequence in the genome (including choloroplastic and mitochondrial DNA) of a plant cell at which a double-strand break is induced in the plant cell genome by a Cas endonuclease. The target site can be an endogenous site in the plant genome, or alternatively, the target site can be heterologous to the plant and thereby not be naturally occurring in the genome, or the target site can be found in a heterologous genomic location compared to where it occurs in nature.

As used herein, terms “endogenous target sequence” and “native target sequence” are used interchangeably herein to refer to a target sequence that is endogenous or native to the genome of a plant and is at the endogenous or native position of that target sequence in the genome of the plant. In an aspect, the target site can be similar to a DNA recognition site or target site that that is specifically recognized and/or bound by a double-strand break inducing agent such as a LIG3-4 endonuclease (US patent publication 2009-0133152 A1 (published May 21, 2009) or a MS26++ meganuclease (U.S. patent application Ser. No. 13/526,912 filed Jun. 19, 2012).

An “artificial target site” or “artificial target sequence” are used interchangeably herein and refer to a target sequence that has been introduced into the genome of a plant. Such an artificial target sequence can be identical in sequence to an endogenous or native target sequence in the genome of a plant but be located in a different position (i.e., a non-endogenous or non-native position) in the genome of a plant.

An “altered target site,” “altered target sequence,” “modified target site,” and “modified target sequence” are used interchangeably herein and refer to a target sequence that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

In an aspect, the disclosed methods and compositions can be used to introduce into dicot plants with increased efficiency and speed polynucleotides useful for the targeted integration of nucleotide sequences into a plant. For example, the disclosed methods and compositions can be used to introduce transfer cassettes comprising nucleotide sequences of interest flanked by non-identical recombination sites to transform a plant comprising a target site. In an aspect, the target site contains at least a set of non-identical recombination sites corresponding to those on the transfer cassette. The exchange of the nucleotide sequences flanked by the recombination sites is affected by a recombinase. Thus, the disclosed methods and compositions can be used for the introduction of transfer cassettes for targeted integration of nucleotide sequences, wherein the transfer cassettes which are flanked by non-identical recombination sites recognized by a recombinase that recognizes and implements recombination at the nonidentical recombination sites. Accordingly, the disclosed methods and composition can be used to improve efficiency and speed of development of dicot plants, derived from immature cotyledon, containing non-identical recombination sites.

In an aspect, the present disclosure comprises methods and compositions for producing a transgenic dicot plant, wherein the method comprises (a) infecting and co-cultivating a dicot immature cotyledon in M5 medium with a bacteria containing a plasmid, (b) recovering transformed immature cotyledons in S30 medium without a selection agent, (c) selecting the transformed immature cotyledon in S30 medium with a selection agent, (d) obtaining transformed embryogenic callus from the immature cotyledon, and (e) regenerating transformed plants from the embryogenic callus, wherein transformation further comprises transforming an immature cotyledon wherein the plasmid comprises a transfer cassette comprising a nucleotide sequence of interest flanked by nonidentical recombination sites; and wherein the immature cotyledon is derived from a plant with a genome comprising a target site flanked by non-identical recombination sites which correspond to the flanking sites of the transfer cassette. The method can further comprise providing a recombinase that recognizes and implements recombination at the nonidentical recombination sites, the recombinase being provided to the immature cotyledon.

Thus, the disclosed methods and compositions can further comprise compositions and methods for the directional, targeted integration of exogenous nucleotides into a transformed plant are provided. In an aspect, the disclosed methods use novel recombination sites in a gene targeting system which facilitates directional targeting of desired genes and nucleotide sequences into corresponding recombination sites previously introduced into the target plant genome.

In an aspect, a nucleotide sequence flanked by two non-identical recombination sites is introduced into an immature cotyledon derived from the target organism's genome establishing a target site for insertion of nucleotide sequences of interest. Once a stable plant or cultured tissue is established a second construct, or nucleotide sequence of interest, flanked by corresponding recombination sites as those flanking the target site, is introduced into the stably transformed plant or plant tissues in the presence of a recombinase protein. This process results in exchange of the nucleotide sequences between the non-identical recombination sites of the target site and the transfer cassette.

It is recognized that the transformed plant prepared in this manner may comprise multiple target sites; i.e., sets of non-identical recombination sites. In this manner, multiple manipulations of the target site in the transformed plant are available. Target site in the transformed plant is intended to mean a DNA sequence that has been inserted into the transformed plant's genome and comprises non-identical recombination sites.

Examples of recombination sites for use in the disclosed method are known in the art and include FRT sites (See, for example, Schlake and Bode (1994) Biochemistry 33: 12746-12751; Huang et al. (1991) Nucleic Acids Research 19: 443-448; Paul D. Sadowski (1995) In Progress in Nucleic Acid Research and Molecular Biology vol. 51, pp. 53-91; Michael M. Cox (1989) In Mobile DNA, Berg and Howe (eds.) American Society of Microbiology, Washington D. C., pp. 116-670; Dixon et al. (1995) 18: 449-458; Umlauf and Cox (1988) The EMBO Journal 7: 1845-1852; Buchholz et al. (1996) Nucleic Acids Research 24: 3118-3119; Kilby et al. (1993) Trends Genet. 9: 413-421: Rossant and Geagy (1995) Nat. Med. 1: 592-594; Albert et al. (1995) The Plant J. 7: 649-659: Bayley et al. (1992) Plant Mol. Biol. 18: 353-361; Odell et al. (1990) Mol. Gen. Genet. 223: 369-378; and Dale and Ow (1991) Proc. Natl. Acad. Sci. USA 88: 10558-105620; all of which are herein incorporated by reference.); Lox (Albert et al. (1995) Plant J. 7: 649-659; Qui et al. (1994) Proc. Natl. Acad. Sci. USA 91: 1706-1710; Stuurman et al. (1996) Plant Mol. Biol. 32: 901-913; Odell et al. (1990) Mol. Gen. Gevet. 223: 369-378; Dale et al. (1990) Gene 91: 79-85; and Bayley et al. (1992) Plant Mol. Biol. 18: 353-361). The two-micron plasmid found in most naturally occurring strains of Saccharomyces cerevisiae, encodes a site-specific recombinase that promotes an inversion of the DNA between two inverted repeats. This inversion plays a central role in plasmid copy-number amplification.

The protein, designated FLP protein, catalyzes site-specific recombination events. The minimal recombination site (FRT) has been defined and contains two inverted 13-base pair (bp) repeats surrounding an asymmetric 8-bp spacer. The FLP protein cleaves the site at the junctions of the repeats and the spacer and is covalently linked to the DNA via a 3′phosphate. Site specific recombinases like FLP cleave and religate DNA at specific target sequences, resulting in a precisely defined recombination between two identical sites. To function, the system needs the recombination sites and the recombinase. No auxiliary factors are needed. Thus, the entire system can be inserted into and function in plant cells. The yeast FLP\FRT site specific recombination system has been shown to function in plants. To date, the system has been utilized for excision of unwanted DNA. See, Lyznik et at. (1993) Nucleic Acid Res. 21: 969-975. In contrast, the present disclosure utilizes non-identical FRTs for the exchange, targeting, arrangement, insertion and control of expression of nucleotide sequences in the plant genome.

In an aspect, a transformed organism of interest, such as an immature cotyledon from a dicot plant, containing a target site integrated into its genome is needed. The target site is characterized by being flanked by non-identical recombination sites. A targeting cassette is additionally required containing a nucleotide sequence flanked by corresponding non-identical recombination sites as those sites contained in the target site of the transformed organism. A recombinase which recognizes the non-identical recombination sites and catalyzes site-specific recombination is required.

It is recognized that the recombinase can be provided by any means known in the art. That is, it can be provided in the organism or plant cell by transforming the organism with an expression cassette capable of expressing the recombinase in the organism, by transient expression, or by providing messenger RNA (mRNA) for the recombinase or the recombinase protein.

By “non-identical recombination sites” it is intended that the flanking recombination sites are not identical in sequence and will not recombine or recombination between the sites will be minimal. That is, one flanking recombination site may be a FRT site where the second recombination site may be a mutated FRT site. The non-identical recombination sites used in the methods of the disclosure prevent or greatly suppress recombination between the two flanking recombination sites and excision of the nucleotide sequence contained therein. Accordingly, it is recognized that any suitable non-identical recombination sites may be utilized in the disclosure, including FRT and mutant FRT sites, FRT and lox sites, lox and mutant lox sites, as well as other recombination sites known in the art.

Suitable non-identical recombination site implies that in the presence of active recombinase, excision of sequences between two non-identical recombination sites occurs, if at all, with an efficiency considerably lower than the recombinationally-mediated exchange targeting arrangement of nucleotide sequences into the plant genome. Thus, suitable non-identical sites for use in the disclosure include those sites where the efficiency of recombination between the sites is low; for example, where the efficiency is less than about 30 to about 50%, preferably less than about 10 to about 30%, more preferably less than about 5 to about 10%.

As noted above, the recombination sites in the targeting cassette correspond to those in the target site of the transformed plant. That is, if the target site of the transformed plant contains flanking non-identical recombination sites of FRT and a mutant FRT, the targeting cassette will contain the same FRT and mutant FRT non-identical recombination sites.

It is furthermore recognized that the recombinase, which is used in the disclosed methods, will depend upon the recombination sites in the target site of the transformed plant and the targeting cassette. That is, if FRT sites are utilized, the FLP recombinase will be needed. In the same manner, where lox sites are utilized, the Cre recombinase is required. If the non-identical recombination sites comprise both a FRT and a lox site, both the FLP and Cre recombinase will be require.

The FLP recombinase is a protein which catalyzes a site-specific reaction that is involved in amplifying the copy number of the two-micron plasmid of S. cerevisiae during DNA replication. FLP protein has been cloned and expressed. See, for example, Cox (1993) Proc. Natl. Acad. Sci. U.S.A 80: 4223-4227. The FLP recombinase for use in the disclosure may be that derived from the genus Saccharomyces. It may be preferable to synthesize the recombinase using plant preferred codons for optimum expression in a plant of interest. See, for example, U.S. Pat. No. 5,929,301, entitled “Novel Nucleic Acid Sequence Encoding FLP Recombinase,” herein incorporated by reference. Novel FRT mutant sites can be used in the practice of the disclosed methods. Such mutant sites may be constructed by PCR-based mutagenesis. Although mutant FRT sites are known (see SEQ ID Nos 2, 3, 4 and 5 of U.S. Pat. No. 5,929,301), it is recognized that other mutant FRT sites may be used in the practice of the disclosure. The present disclosure does not require the use of a particular FRT or recombination site, but rather that non-identical recombination sites or FRT sites can be utilized for targeted insertion and expression of nucleotide sequences in a plant genome. Thus, other mutant FRT sites can be constructed and utilized based upon the present disclosure.

The bacteriophage recombinase Cre catalyzes site-specific recombination between two lox sites. The Cre recombinase is known in the art. See, for example, Guo et al. (1997) Nature 389: 40-46; Abremski et al. (1984) J. Biol. Chem. 259: 1509-1514; Chen et al. (1996) Somat. Cell Mol. Genet. 22: 477-488; and Shaikh et al. (1977) J. Biol. Chem. 272: 5695-5702. All of which are herein incorporated by reference. Such Cre sequence may also be synthesized using plant preferred codons.

As discussed above, bringing genomic DNA containing a target site with non-identical recombination sites together with a vector containing a transfer cassette with corresponding non-identical recombination sites, in the presence of the recombinase, results in recombination. The nucleotide sequence of the transfer cassette located between the flanking recombination sites is exchanged with the nucleotide sequence of the target site located between the flanking recombination sites. In this manner, nucleotide sequences of interest may be precisely incorporated into the genome of the host.

It is recognized that many variations of the disclosure can be practiced. For example, target sites can be constructed having multiple non-identical recombination sites. Thus, multiple genes or nucleotide sequences can be stacked or ordered at precise locations in the plant genome. Likewise, once a target site has been established within the genome, additional recombination sites may be introduced by incorporating such sites within the nucleotide sequence of the transfer cassette and the transfer of the sites to the target sequence. Thus, once a target site has been established, it is possible to subsequently add sites, delete sites, or alter sites through recombination.

Another variation includes providing a promoter or transcription initiation region operably linked with the target site in an organism. Preferably, the promoter will be 5′ to the first recombination site. By transforming the organism with a transfer cassette comprising a coding region, expression of the coding region will occur upon integration of the transfer cassette into the target site. This aspect provides for a method to select transformed cells, particularly plant cells, by providing a selectable marker sequence as the coding sequence.

Other advantages of the present system include the ability to reduce the complexity of integration of transgenes or transferred DNA in an organism by utilizing transfer cassettes as discussed above and selecting organisms with simple integration patterns. In the same manner, preferred sites within the genome can be identified by comparing several transformation events. A preferred site within the genome includes one that does not disrupt expression of essential sequences and provides for adequate expression of the transgene sequence.

Any means known in the art for bringing the three components of the system together may be used in the disclosure. For example, a dicot plant can be stably transformed to harbor the target site in its genome. The recombinase may be transiently expressed or provided. Alternatively, a nucleotide sequence capable of expressing the recombinase may be stably integrated into the genome of the dicot plant. In the presence of the corresponding target site and the recombinase, the transfer cassette, flanked by corresponding non-identical recombination sites, is inserted into the transformed dicot plant's genome.

Alternatively, the components of the system may be brought together by sexually crossing transformed plants. In this aspect, a transformed plant, parent one, containing a target site integrated in its genome can be sexually crossed with a second plant, parent two, that has been genetically transformed with a transfer cassette containing flanking non-identical recombination sites, which correspond to those in plant one. Either plant one or plant two contains within its genome a nucleotide sequence expressing a recombinase. The recombinase may be under the control of a constitutive or inducible promoter.

Inducible promoters include enviromentally inducible heat shock promoters including HSP17.7, HSP26, or HSP18A, as well as, heat-inducible promoters, estradiol-responsive promoters, chemical inducible promoters, and the like. Pathogen inducible promoters include those from pathogenesis-related proteins (PR proteins), which are induced following infection by a pathogen; e. g., PR proteins, SAR proteins, beta-1,3-glucanase, chitinase, etc. See, for example, Redolfi et al. (1983) Neth. J. Plant Pathol. 89: 245-254; Uknes et al. (1992) The Plant Cell 4: 645-656; and Van Loon (1985) Plant Mol. Virol. 4: 111-116. In this manner, expression of recombinase and subsequent activity at the recombination sites can be controlled.

Constitutive promoters for use in expression of genes in plants are known in the art. Such promoters include, but are not limited to 35S promoter of cauliflower mosaic virus (Depicker et al. (1982) Mol. Appl. Genet. 1: 561-573; Odell et al. (1985) Nature 313: 810-812), ubiquitin promoter (Christensen et al. (1992) Plant Mol. Biol. 18: 675-689), promoters from genes such as ribulose bisphosphate carboxylase (De Almeida et al. (1989) Mol. Gen. Genet. 218: 78-98), actin (McElroy et al. (1990) Plant J. 2: 163-171), histone, DnaJ (Baszczynski et al. (1997) Maydica 42: 189-201), and the like.

The disclosed compositions and methods are useful in targeting the integration of transferred nucleotide sequences to a specific chromosomal site. The nucleotide sequence may encode any nucleotide sequence of interest. Particular genes of interest include those which provide a readily analyzable functional feature to the host cell and/or organism, such as marker genes, as well as other genes that alter the phenotype of the recipient cells, and the like. Thus, genes effecting plant growth, height, susceptibility to disease, insects, nutritional value, and the like may be utilized in the disclosure. The nucleotide sequence also may encode an ‘antisense’ sequence to turn off or modify gene expression.

It is recognized that the nucleotide sequences will be utilized in a functional expression unit or cassette. Functional expression unit or cassette is intended to mean, the nucleotide sequence of interest with a functional promoter, and in most instances a termination region. There are various ways to achieve the functional expression unit within the practice of the disclosure. In one aspect of the disclosure, the nucleic acid of interest is transferred or inserted into the genome as a functional expression unit.

Alternatively, the nucleotide sequence may be inserted into a site within the genome which is 3′ to a promoter region. In this latter instance, the insertion of the coding sequence 3′ to the promoter region is such that a functional expression unit is achieved upon integration. For convenience, for expression in dicot plants, the nucleic acid encoding target sites and the transfer cassettes, including the nucleotide sequences of interest, can be contained within expression cassettes. The expression cassette will comprise a transcriptional initiation region, or promoter, operably linked to the nucleic acid encoding the peptide of interest. Such an expression cassette is provided with a plurality of restriction sites for insertion of the gene or genes of interest to be under the transcriptional regulation of the regulatory regions.

The transcriptional initiation region, the promoter, may be native or homologous or foreign or heterologous to the host plant, or could be the natural sequence or a synthetic sequence. By foreign it is intended that the transcriptional initiation region is not found in the wild-type host plant into which the transcriptional initiation region is introduced. Either a native or heterologous promoter may be used with respect to the coding sequence of interest.

The transcriptional cassette will include in the 5′-3′ direction of transcription, a transcriptional and translational initiation region, a DNA sequence of interest, and a transcriptional and translational termination region functional in dicot plants. The termination region may be native with the transcriptional initiation region, may be native with the DNA sequence of interest, or may be derived from another source. Convenient termination regions are available from the potato proteinase inhibitor (PinII) gene or sequences from Ti-plasmid of A. tumefaciens, such as the nopaline synthase, octopine synthase and opaline synthase termination regions. See also, Guerineau et al., (1991) Mol. Gen. Genet. 262: 141-144; Proudfoot (1991) Cell 64: 671-674; Sanfacon et al. (1991) Genes Dev. 5: 141-149; Mogen et al. (1990) Plant Cell 2: 1261-1272; Munroe et al. (1990) Gene 91: 151-158; Ballas et al. 1989) Nucleic Acids Res. 17: 7891-7903; Joshi et al. (1987) Nucleic Acid Res. 15: 9627-9639.

Where appropriate, the nucleotide sequences to be inserted in the plant genome may be optimized for increased expression in the transformed plant. Where mammalian, yeast, or bacterial genes are used in the disclosure, they can be synthesized using plant preferred codons for improved expression. It is recognized that for expression in dicots, monocot genes can also be synthesized using dicot preferred codons. Methods are available in the art for synthesizing plant preferred genes. See, for example, U.S. Pat. Nos. 5,380,831 and 5,436,391, and Murray et al. (1989) Nucleic Acids Res. 17: 477-498, herein incorporated by reference. The plant preferred codons may be determined from the codons utilized more frequently in the proteins expressed in the plant of interest. It is recognized that monocot or dicot preferred sequences may be constructed as well as plant preferred sequences for particular plant species. See, for example, EPA 0359472; EPA 0385962; WO 91/16432; Perlak et al. (1991) Proc. Natl. Acad. Sci. USA, 88: 3324-3328; and Murray et al. (1989) Nucleic Acids Research, 17: 477-498. U.S. Pat. Nos. 5,380,831; 5,436,391; and the like, herein incorporated by reference. It is further recognized that all or any part of the gene sequence may be optimized or synthetic. That is, fully optimized or partially optimized sequences may also be used.

Additional sequence modifications are known to enhance gene expression in a cellular host. These include elimination of sequences encoding spurious polyadenylation signals, exon-intron splice site signals, transposon-like repeats, and other such well-characterized sequences which may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The methods disclosed herein are useful in regulating expression of any heterologous nucleotide sequence in a host plant in order to vary the phenotype of a plant. As will be evident to one of skill in the art, any nucleic acid of interest can be used in the methods of the disclosure. For example, a soybean plant can be engineered to express disease and insect resistance genes, genes conferring nutritional value, genes to confer male and/or female sterility, antifungal, antibacterial or antiviral genes, and the like. Likewise, the method can be used to transfer any nucleic acid to control gene expression. For example, the nucleic acid to be transferred could encode an antisense oligonucleotide.

Various changes in phenotype are of interest including modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, and commercial products.

Agronomically important traits such as oil, protein content, and the like can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802 and 5,990,389, herein incorporated by reference. Another example is lysine and/or sulfur rich seed protein encoded by the soybean 2S albumin described in U.S. Pat. No. 5,850,016, and the chymotrypsin inhibitor from barley, described in Williamson, et al., (1987) Eur. J. Biochem. 165:99-106, the disclosures of which are herein incorporated by reference.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881 and Geiser, et al., (1986) Gene 48:109), and the like.

Genes encoding disease resistance traits include detoxification genes, such as against fumonosin (U.S. Pat. No. 5,792,931); avirulence (avr) and disease resistance (R) genes (Jones, et al., (1994) Science 266:789; Martin, et al., (1993) Science 262:1432 and Mindrinos, et al., (1994) Cell 78:1089), and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene); glyphosate (e.g., the EPSPS gene and the gat gene; see, for example, US Patent Application Publication Number 2004/0082770 and WO 2003/092360) or other such genes known in the art. The bar gene encodes resistance to the herbicide basta, the nptII gene encodes resistance to the antibiotics kanamycin and geneticin, and the ALS-gene mutants encode resistance to the herbicide chlorsulfuron.

The concentration of a disarmed Agrobacteria, an Ochrobactrum bacteria or a Rhizobiaceae bacteria useful in the methods of the disclosure may vary depending on the disarmed Agrobacteria, the Ochrobactrum bacteria or the Rhizobiaceae bacteria strain utilized, the tissue being transformed, the plant genotype being transformed, and the like. While the concentration of the disarmed Agrobacteria, the Ochrobactrum bacteria or the Rhizobiaceae bacteria strain may vary, generally a concentration range of about 1×10³ cfu/ml to about 1×10¹⁰ cfu/ml, preferably within the range of about 1×10³ cfu/ml to about 1.5×10⁹ cfu/ml, and still more preferably at about 0.5×10⁹ cfu/ml to about 1.0×10⁹ cfu/ml, will be utilized.

In some cases, the tissue to be contacted with a disarmed Agrobacteria, an Ochrobactrum bacteria or a Rhizobiaceae bacteria strain is an immature cotyledon. Embryogenic callus can originate in any appropriate tissue of a dicot plant. Preferably, tissue utilized in initiating callus is immature tissue such as immature cotyledons, immature embryos, immature inflorescences, and the basal portion of young leaves. For example, primary or secondary embryogenic callus can be excised from immature cotyledons. In some cases, the tissue can be wounded or chopped prior to or simultaneously with contact with a disarmed Agrobacteria, an Ochrobactrum bacteria or a Rhizobiaceae bacteria strain. For example, plant tissue can be wounded by chopping, cutting, sonication or some other means prior to contacting the tissue with a disarmed Agrobacteria, an Ochrobactrum bacteria or a Rhizobiaceae bacteria strain comprising a vector which comprises a nucleotide sequence of interest.

In the present disclosure, an immature cotyledon may be of varying lengths. Immature cotyledon length may be from 0.5 mm to 9.0 mm including, but not limited to, any length between 0.5 mm and 9.0 mm, for example, 0.5, 1, 1.5, 2, 2.5, 3, 3.5, 4, 4.5, 5, 5.5, 6, 6.5, 7, 7.5, 8.0, 8.5, and 9.0 mm. The range of 0.5 mm to 9.0 mm includes any value including, and between, 0.5 mm and 9.0 mm, such as for example 0.51, 0.52, 0.53, 0.54, 0.55, 0.56, 0.57, etc.

Bacterial infection of the immature cotyledon may occur through various methods well known in the art. The infection process may be enhanced by wounding either at the time of infection or prior to infection. Wounding may occur through sonication, through the aid of an abrasive material, by vortexing, by shaking, or by particle bombardment.

The immature cotyledons are co-cultivated with a disarmed Agrobacteria, an Ochrobactrum bacteria or a Rhizobiaceae bacteria strain in the presence of a culture medium provided herein. As used interchangeably herein, “co-cultivating”, “co-cultivation”, “co-culture” and “infection” refer to incubating a disarmed Agrobacteria, an Ochrobactrum bacteria or a Rhizobiaceae bacteria-contacted/infected plant tissue in the presence of the cultivation medium described herein to allow continued T-DNA delivery from a disarmed Agrobacteria, an Ochrobactrum bacteria or a Rhizobiaceae bacteria into plant cells.

In some aspects, co-cultivation of plant tissue with a disarmed Agrobacteria, an Ochrobactrum bacteria or a Rhizobiaceae bacteria can take place on a porous solid support in the presence of the cultivation medium of the disclosure. For example, co-cultivation can take place with plant tissue to be transformed placed on a porous solid support (e.g., filter paper, glass fiber filter). Any appropriate porous solid support that prevents disarmed Agrobacteria, Ochrobactrum bacteria or Rhizobiaceae bacteria overgrowth and retains moisture and nutritional elements can be used according to the methods provided herein. Exemplary porous solid supports can include VWR grade 415 filter paper, Whatman grade 1 filter paper, and VWR grade 693 glass fiber filters.

Immature cotyledons or other plant tissue can be positioned in any appropriate orientation for co-cultivation in the presence of the cultivation medium. By way of example, and without limitation, immature cotyledons can be face up or face down.

The immature cotyledons or other plant tissue can be co-cultivated with a disarmed Agrobacteria, an Ochrobactrum bacteria or a Rhizobiaceae bacteria strain for about 1-30 days, preferably about 2-20 days and more preferably about 3-7 days.

In some aspects, the methods provided herein can further include the step of culturing or incubating the tissue (for example, soybean immature cotyledon) for a length of time prior to the bacteria-mediated or biolistic-mediated transformation in a pre-culturing or pre-incubating step. “Pre-culturing”, “pre-cultured”, “pre-incubating”, and “pre-incubated” as used herein means culturing or incubating the cells or tissues in an appropriate pre-culture/pre-incubation medium to support plant tissue growth prior to the introduction of a nucleic acid. In some aspects, tissue is pre-cultured/pre-incubated for several days (e.g., about 1 day, about 2 days, about 3 days, about 4 days, about 5 days, or more). Pre-culturing/pre-incubating the plant cells may be performed using any method known to one ordinarily skilled in the art. In some cases, pre-culturing/pre-incubating can be performed in a cultivation/incubation medium containing glutamine and L-methionine as provided herein. In some cases, the pre-culturing/pre-incubating is performed in S30 medium.

Following the co-cultivation step, the transformed cells may be subjected to an optional resting step. As used herein, “resting” refers to a culture step where plant cells, such as immature cotyledons, or other tissue, are incubated after the introduction of the nucleic acid by a disarmed Agrobacteria, an Ochrobactrum bacteria or a Rhizobiaceae bacteria-mediated infection. The resting step permits the preferential initiation and growth of callus from the transformed cells containing the nucleic acid of interest and is usually carried out in the absence of any selective pressures. The transformed plant tissue is subjected to a resting media that typically includes an antibiotic capable of inhibiting the disarmed Agrobacteria, the Ochrobactrum bacteria or the Rhizobiaceae bacteria growth. Such antibiotics are known in the art and include cefotaxime, timentin (ticarcillin disodium and clavulanate potassium), vancomycin, carbenicillin, Plant Preservative Mixture™ (Plant Cell Technology, Inc., Washington, D.C.), and the like. Concentrations of the antibiotic will vary according to what is standard for each antibiotic. For example, concentrations of timentin will range from about 50 mg/L to about 500 mg/L, for carbenicillin in solid media, preferably about 75 mg/L to about 500 mg/L, and more preferably about 100-300 mg/L. Those of ordinary skill in the art of dicot transformation will recognize that the concentration of antibiotic can be optimized for a particular transformation protocol without undue experimentation.

In some aspects, the resting phase cultures are allowed to rest in the light at 26° C. for about 1 to about 15 days, preferably for about 3 to about 10 days, more preferably for about 5 to about 8 days. In some aspects, conditions for the resting step can be those conditions exemplified herein.

Where no resting step is used, an extended co-cultivation step can be used to provide a period of culture time prior to the addition of a selective agent for the transformed cells.

The methods provided herein further include selecting regenerable cells comprising a nucleotide sequence of interest. “Selecting” as used herein refers to the culture step in plant transformation where the transformed cells that have received and are expressing a selection marker from the introduced nucleic acid are selected. Following the co-cultivation step, or following the resting step, where it is used, the transformed cells can be exposed to selective pressure to select for those cells that have received and are expressing a polypeptide from the heterologous nucleic acid introduced by a disarmed Agrobacteria, an Ochrobactrum bacteria or a Rhizobiaceae bacteria or bv. In some cases, cells may be exposed to a selective pressure in order to favor those cells that express the selection marker and may include the use of a selective agent that allows for selection of transformants containing at least one selection marker insert. For example, where the cells are immature cotyledons, the immature cotyledons can be transferred to plates with solid medium or the flasks with liquid medium that includes both an antibiotic to inhibit growth of the disarmed Agrobacteria, the Ochrobactrum bacteria or th Rhizobiaceae bacteria and a selection agent or in the case of biolistic-mediated transformation the medium is supplemented with only a selection agent. The selection agent used to select for transformants will select for preferential growth of explants containing at least one selectable marker present in a DNA or ribonucleoprotein biolistically delivered in the case of biolistic-mediated transformation or from an insert positioned within the super binary vector and delivered by a disarmed Agrobacteria, an Ochrobactrum bacteria or a Rhizobiaceae bacteria.

Any suitable selection marker may be used including, without limitation, hygromycin, bar, pat, gat, ALS, PMI, hpt, nptII, and positive and/or negative selectable markers and visible selection marker genes such as DS-RED, GFP, YFP, GUS and the like. Any suitable selective agent may be used including, without limitation, herbicides, such as, a sulfonylurea herbicide, an imidazolinone herbicide, bialaphos, phosphinothricin, glyphosate, glufosinate-NH4 (PPT), chlorsulfuron, ethametsulfuron, imazapyr, sugar, such as mannose, and antibiotics such as hygromycin B or G418, spectinomycin, kanamycin, and the like or a combination thereof. During the selecting step, dead and necrotic tissues can be discarded, and green embryogenic callus can be subcultured to fresh medium containing the selection agent.

Selecting may optionally be carried out in light, dim, or dark conditions. The length of exposure of the plant cell to light, dim, or dark conditions may vary based in part on the type of plant species and genotype being transformed. Preferably, plant cells are rested and selected in light conditions. In some aspects, conditions for the selection step can be those conditions exemplified in Example 3.

After transformed plant tissue has been identified and selected, the transformed tissue can be regenerated into whole plants. Any appropriate method of regenerating whole plants can be used. The regeneration, development, and cultivation of plants from various transformed explants, including but not limited to, embryogenic callus, immature and mature embryo, cotyledons and immature cotyledons, immature and mature seed, meristem, cell clusters, scutella, nodes, young leaf bases, hypocotyl explants, roots, embryonic axis, inflorescences, suspension cultures, cultures of suspended cell aggregates, meristematic regions, leaves, green tissue, non-green tissue, somatic embryos and shoot apexes are well known in the art. See, for example, US Patent Publication 2018/0216123; Finer and Naganawa, (1998) Plant Cell Tissue Org. Cult. 15:125-136; and Samoylov, et al., (1998) In Vitro Cellular and Developmental Biology—Plant 34:8-13; McCormick et al. (1986) Plant Cell Reports 5:81-84; Weissbach and Weissbach, In: Methods for Plant Molecular Biology, (Eds.), Academic Press, Inc. San Diego, Calif., (1988), all of which are herein incorporated by reference in their entireties. This regeneration and growth process typically includes the steps of selection of transformed cells, culturing those individualized cells through the usual stages of embryonic development through the rooted plantlet stage. In some cases, transformed embryogenic callus tissue or other transformed plant tissue can be subcultured at regular or irregular intervals in the same medium. Transgenic embryos and seeds are similarly regenerated. Individual calli can be individually separated to ensure that only one whole plant is regenerated per callus and, therefore, that all regenerated plants are derived from independent transformation events. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a polynucleotide of the disclosure, for example, an expression cassette of the disclosure, stably incorporated into their genome.

The methods and compositions provided herein can be used to produce regenerable plant cells with reduced incidence of necrosis. By “reduced incidence of necrosis” is intended that transformed plant tissue exhibits fewer or smaller necrotic lesions or other indicators of plant tissue necrosis. In some cases, “reduced incidence of necrosis” can be determined relative to plant tissue transformed in the absence of a composition provided herein. Necrosis in transformed plant tissue can be detected by physically assessing the appearance of transformed tissue and, in some cases, quantifying (i.e., measuring the number and/or diameter of) necrotic lesions on the surface of the transformed tissue. For example, the extent of necrosis can be readily quantified by plant biologists and technicians through visual assessment of the area of any necrotic lesions relative to the total surface area of the plant tissue following Agrobacterium-mediated transformation. In some aspects, a decrease in tissue necrosis (i.e., decrease in lesion diameter or number of lesions) can be observed in tissues transformed according to the methods provided herein. In the practice of the disclosure, following the co-cultivation step, necrotic lesions can, on average, account for no more than about 30% of the total area of tissue transformed according to the methods provided herein. Thus, following the co-cultivation step, non-necrotic tissue can, on average, account for more than about 70% (e.g., about 70%, about 75%, about 80%, about 85%, about 90%, about 95%, about 99%) of the total area of tissue transformed according to the methods provided herein.

The methods described herein provide for an efficient method of increasing the transformation of dicots. Any suitable dicot may be used with the methods and compositions described herein. These include, without limitation, soybean (e.g., Glycine max), Brassica spp. (e.g., B. napus, B. rapa, B. juncea), particularly those Brassica species useful as sources of seed oil, sunflower (e.g., Helianthus annuus), cotton (e.g., Gossypium barbadense, Gossypium hirsutum) or alfalfa (e.g., Medicago sativa), or kale, or cauliflower, or broccoli, or pea, or clover, or broad bean, or tomato, or cassava, or peanut, or safflower, or tobacco, or Arabidopsis and the like.

The article “a” and “an” are used herein to refer to one or more than one (i.e., to at least one) of the grammatical object of the article. By way of example, “an element” means one or more element.

Throughout the specification, the word “comprising,” or variations such as “comprises,” will be understood to imply the inclusion of a stated element, integer or step, or group of elements, integers or steps, but not the exclusion of any other element, integer or step, or group of elements, integers or steps.

The following examples are presented by way of illustration, and not by way of limitation.

### EXAMPLES

### Example 1. Plant Materials and Media Compositions

The methods used to generate soybean embryogenic cultures are described by Finer and Nagasawa (Plant Cell Tiss. Org. Cult. 15:125-136 (1988)), Samoylov et al. (In Vitro Cell. Dev. Biol.-Plant 34: 8-13 (1998)) and in U.S. Pat. No. 8,962,328, all of which are herein incorporated by reference in their entirety.

Immature seeds 2-4 mm in length were harvested from immature soybean pods of plants grown in the greenhouse under standard conditions. The techniques used throughout these Examples can be applied to a wide range of soybean cultivars and other dicotyledonous plants.

The compositions of various medium used in this disclosure are outlined in Table 1.

TABLE 1
Composition of media.

| | M2 | | M5 | | Base | | S30 | | M7 | | M8 | |
|---|---|---|---|---|---|---|---|---|---|---|---|---|
| MS salt with B5 | | | | | | | | | 4.44 | g/L | | |
| vitamins (PhytoTech | | | | | | | | | | | | |
| M404) | | | | | | | | | | | | |
| Gamborg B-5 basal | | | | | | | | | | | 3.21 | g/L |
| medium (PhytoTech | | | | | | | | | | | | |
| G398) | | | | | | | | | | | | |
| Modified MS salt | 2.68 | g/L | 2.68 | g/L | 2.68 | g/L | 2.68 | g/L | | | | |
| (PhytoTech M571) | | | | | | | | | | | | |
| B5 vitamins | 1 | ml | 1 | ml | 1 | ml | 1 | ml | | | | |
| (1000X) | | | | | | | | | | | | |
| (PhytoTech G249) | | | | | | | | | | | | |
| 2,4-D stock 10 | 1 | ml | 1 | ml | | | 1 | ml | | | | |
| mg/ml | | | | | | | | | | | | |
| KNO₃ | 0.93 | g/L | 1.64 | g/L | 0.93 | g/L | 0.93 | g/L | | | | |
| (NH₄)₂SO₄ | 0.463 | g/L | 0.463 | g/L | 0.463 | g/L | 0.463 | g/L | | | | |
| Asparagine | 1 | g/L | 1 | g/L | 1 | g/L | 1 | g/L | | | | |
| Glutamine | | | | | 4.48 | g/L | 4.48 | g/L | | | | |
| L-Methionine | | | | | 0.149 | g/L | 0.149 | g/L | | | | |
| Sucrose | 10 | g/L | 68.5 | g/L | 10 | g/L | 10 | g/L | | | 10 | g/L |
| Glucose | | | 36 | g/L | | | | | | | | |
| Sorbitol | | | | | | | | | | | | |
| Mannitol | | | | | | | | | | | | |
| Maltose | | | | | | | | | 60 | g/L | | |
| MgCl₂.6H₂O | | | | | | | | | 0.75 | g/L | | |
| Activated charcoal | | | | | | | | | 5 | g/L | | |
| (PhytoTech C325) | | | | | | | | | | | | |
| Casein hydrolysate | | | 1 | g/L | | | | | | | | |
| (PhotoTech C184) | | | | | | | | | | | | |
| pH | 5.8 | | 5.4 | | 5.8 | | 5.8 | | 5.7 | | 5.7 | |
| Acetosyringone | | | 200 | μM | | | | | | | | |
| DTT | | | 1 | mM | | | | | | | | |
| TC agar | | | | | | | | | | | 5 | g/L |
| Gelrite (Plant Media | | | | | | | | | | | | |
| Cat# 714246) | | | | | | | | | 2 | g/L | | |

### Example 2. Agrobacterium Strains and Plasmids

Agrobacterium tumefaciens strains AGL-1, EHA105, GV3101, and LBA4404 containing a binary plasmid were used. Two binary test plasmids were used. Binary test plasmid A contained the coding region for an intron-containing the ZS-Yellow fluorescent protein (YFP) gene driven by the H2B promoter and a hygromycin resistance gene driven by the CaMV35S promoter within the T-DNA borders. Binary test plasmid B contained the coding region for an intron-containing the Tag red fluorescent protein (TagRFP) gene driven by the Arabidopsis UBQ10 promoter and terminated by the GM-UBQ3 terminator and an intron-containing the GM-HRA gene driven by the GM SAMS promoter within the T-DNA borders. The use of fluorescent proteins, such as YFP and TagRFP, provided noninvasive detection of expression in living cells without the use of additional substrates. Real time visualization of gene expression was therefore observed. The plasmids (binary test plasmid A and binary test plasmid B) were introduced into the Agrobacterium strains (AGL-1, EHA105, GV3101, and LBA4404) by electroporation and cultured for 2-3 days on LB agar plates (10 g/L tryptone, 5 g/L yeast extract, 5 g/L NaCl, 8 g/L agar, see also, Sambrook J, Fritsch E F, Maniatis T (1989) Molecular cloning: a laboratory manual. Cold Spring Harbor Laboratory Press, New York) supplemented with 100 mg/L kanamycin at 28° C. to provide Kanamycin-resistant colonies. Kanamycin-resistant colonies were then grown to an OD600 nm of 1.0-1.5 in LB liquid medium supplemented with 100 mg/L kanamycin and frozen glycerol stock cultures were prepared and stored at −80° C. for future use.

### Example 3. Transformation and Plant Regeneration Procedures

The day before transformation, the A. tumefaciens strains (AGL-1, EHA105, GV3101, and LBA4404) containing plasmids (binary test plasmid A and binary test plasmid B) from the glycerol stock were inoculated in a tube containing 5 ml of fresh LB liquid medium supplemented with 100 mg/L kanamycin, and then placed on a shaker incubator at 250 rpm overnight at 28° C. On the day of transformation, log phase A. tumefaciens AGL-1, EHA 105, GV3101 or LBA4404 cells containing the binary test plasmid (A or B) were centrifuged at 1,500×g for 10 minutes and cell pellets were resuspended in liquid M5 infection/co-cultivation medium (see Table 1 for M5 medium composition). The Agrobacterium was diluted to an OD of 0.5 at 600 nm with co-cultivation M5 medium and used as the inoculum. Agrobacterium at an OD of from 0.02 to 1.5 at 600 nm provided similar results (data not shown).

Immature soybean seeds were surface sterilized in a 50 mL screw cap tube containing 50 mL of a 10% bleach, 0.02% Tween-20 solution, with slight agitation for 15 minutes and were then rinsed 10 times with a total of 500 mL of sterile distilled water. Immature cotyledons were aseptically excised by cutting the embryo axis off the cotyledons and then pushing the cotyledons out of the seed coat onto sterile 7.5 cm filter paper moistened with sterile distilled water in a deep petri dish (25×100 mm). 20-25 isolated immature cotyledons were transferred into a sterile glass tube (16×100 mm) containing 400 μL of the Agrobacterium inoculums. Sonication (1 second) was performed in a sonic water bath (VWR 50T). After sonication, the immature cotyledons were left in the inoculum for 15 minutes at room temperature for infection. After 15 minutes of infection, immature cotyledons from two glass tubes were poured onto double layered sterile filter papers (total 800 μl/double layered filter) in a deep petri dish and then the petri dishes were wrapped with two layers of Parafilm for co-cultivation for 4 days at 21° C. in a Percival brand incubator at a light intensity of 3-5 μE/m²/s. During the co-cultivation period, the levels of transient expression of TagRFP or YFP were observed under a fluorescence stereomicroscope equipped with an appropriate filter set for excitation and emission. Tissue viability was also observed.

After 4 days of co-cultivation, immature cotyledons were washed off of the filter paper with S30 medium (see Table 1 for S30 medium composition) supplemented with 300 μg/mL timentin antibiotic and were rinsed three times to remove residual Agrobacterium. The immature cotyledons were then transferred to a 250 mL sterile glass flask (40-50 immature cotyledons/flask) containing 40-50 mL S30 medium supplemented with 300 mg/L timentin antibiotic to kill the Agrobacterium without selection, and were cultured at 25-26° C. with an 18-hour photoperiod at 35-60 μE/m²/s light intensity for 7 days on rotary shaker at 100 rpm for the recovery period.

Following the recovery period, selection agents chlorsulfuron or hygromycin were used for the selection of stable transformants. The recovery medium was replaced with 40-50 mL S30 medium supplemented with 300 mg/L timentin or 10 μg/L chlorsulfuron (Chem Service, West Chester, Pa., USA) or 300 mg/L timentin or 15 mg/L hygromycin (CalBiochem, La Jolla, Calif., USA) for the selection of transformed cells. The selection medium was replaced bi-weekly and cultured at 25-26° C. with 18-hour photoperiod at 35-60 μE/m²/s light intensity on a rotary shaker at 100 rpm. After 4-8 weeks on selection medium, transformed tissue became visible as green tissue against a background of bleached, less healthy tissue.

Putative transformed green callus was isolated under a microscope and plated onto petri plates with sterile filter paper overlaying M7 agar medium (see Table 1 for M7 medium composition) supplemented with 300 mg/L timentin for embryo maturation. The petri plates were sealed with Micropore surgical tape (3M Health Care, St. Paul, Minn., USA) and incubated at 26° C. with an 18-hour photoperiod at 35-60 μE/m²/s light intensity. After 3-4 weeks of maturation on M7 medium, mature somatic embryos were placed in sterile, Petri dishes and either sealed with Micropore™ surgical tape or placed unsealed in a plastic box for 4-7 days at room temperature for somatic embryo desiccation. After 4-7 days, desiccated embryos were plated onto M8 medium (see Table 1 for M8 medium composition) supplemented with 5 μg/L chlorsulfuron or 15 mg/L hygromycin and were allowed to germinate at 26° C. with an 18-hour photoperiod at 35-60 μE/m²/s light intensity. After 4-6 weeks on M8 germination medium, plantlets were transferred to 4 inch pots containing moistened Berger BM2 soil (Berger Peat Moss, Saint-Modeste, Canada) and kept enclosed in clear plastic tray boxes until acclimatized in a culture room with a 16-hour photoperiod at 90-150 μE/m²/s at 26° C. day/24° C. night temperatures. After acclimation, hardened plantlets were potted in 2 gallon pots containing moistened Berger MB1 (Berger Peat Moss, Saint-Modeste, Canada) and grown in a greenhouse to seed-bearing maturity.

This example demonstrated Agrobacterium-mediated transformation of soybean immature cotyledon. Transgenic T0 events were produced within 3.5-4.5 months after transformation was initiated.

### Example 4. The Effect of Glutamine and L-Methionine in Embryogenic Callus Induction from Immature Cotyledons

The influence of L-methionine and glutamine on the induction of embryogenic callus in the presence of S30 medium was evaluated. Two (2) to four (4) mm lengths of immature cotyledons of elite soybean cultivars A-D were cultured in M2 medium and in S30 medium which is M2 medium supplemented with glutamine (4.48 g/L (30.6 mM)) and L-methionine (0.149 g/L (1 mM)). See Table 1. The same culture conditions described above were used in this Example 4. The induction of highly regenerable green, healthy embryogenic callus was dramatically increased when immature cotyledons from elite cultivars A-D were cultured in S30 medium. The induction of green embryogenic callus was observed after 7-10 days of culture in S30 medium. Conversely, immature cotyledons cultured on M2 medium (without glutamine and L-methionine) showed very poor induction of embryogenic callus or showed bleaching of tissues within 1-2 weeks of culture as shown in FIG. 1.

To further evaluate the effects of medium supplemented with glutamine and L-methionine on the induction of embryogenic callus, five experiments were conducted using different medium formulation treatments. See Table 1 for M2 medium composition. The five medium formulation treatments were as follows:

Treatment 1—M2 medium;

Treatment 2—M2 medium supplemented with 1 mM L-methionine;

Treatment 3—M2 medium supplemented with 1 mM L-methionine and 1 mM glutamine;

Treatment 4—M2 medium supplemented with 1 mM L-methionine and 30.6 mM glutamine (S30 medium); and

Treatment 5—M2 medium supplemented with 30.6 mM glutamine.

The results are shown in FIG. 2A-FIG. 2E. As shown in FIG. 2E, 30.6 mM glutamine significantly increased the induction of embryogenic callus formation. Furthermore, as shown in FIG. 2D, the addition of 1 mM L-methionine resulted in further induction of embryogenic callus formation.

### Example 5. Agrobacterium-Mediated Soybean Immature Cotyledon Transformation Using Hygromycin Selection in M2 Medium or S30 Medium

Immature cotyledons of soybean elite variety 93Y21 were transformed with A. tumefaciens AGL-1 harboring the test binary plasmid A described in Example 2. Four days after co-cultivation in M5 medium, transformed immature cotyledons were cultured on M2 medium or S30 medium each supplemented with 300 mg/L timentin without a selection agent for during recovery. After 1-week of recovery, immature cotyledons were cultured for 6 weeks on M2 medium or S30 medium each supplemented with 300 mg/L timentin and 15 mg/L hygromycin. Fresh media was replaced bi-weekly.

Immature cotyledons cultured on M2 medium showed only one embryogenic callus out of 49 immature cotyledons transformed resulting in a transformation frequency of only 2%. In contrast, immature cotyledons cultured on S30 medium showed 16 green embryogenic calli out of 74 immature cotyledons transformed resulting in a transformation frequency of 22% which is a 10-fold increase in transformation frequency compared to the transformation frequency of culturing on M2 medium. See Table 2.

TABLE 2
The effect of glutamine and L-methionine (S30 medium) on
transformation frequency of soybean using hygromycin selection

| | Number of immature | Number of green embryogenic |
|---|---|---|
| | cotyledons | callus and (% transformation |
| Medium | transformed | frequency) |
| M2 | 49 | 1 (2%) |
| S30 | 74 | 16 (22%) |

### Example 6. Agrobacterium-Mediated Soybean Immature Cotyledon Transformation Using Chlorsulfuron Selection in M2 or S30 Medium

Immature cotyledons of soybean elite cultivar 93Y21 were transformed with A. tumefaciens AGL-1 harboring the binary test plasmid B described in Example 2. Four days after co-cultivation in M5 medium, transformed immature cotyledons were cultured on M2 medium or S30 medium each supplemented with 300 mg/L timentin without a selection agent for recovery. After 1-week recovery, immature cotyledons were cultured for 6 weeks on M2 medium or S30 medium each supplemented with 300 mg/L timentin 300 mg/L and 10 μg/L chlorsulfuron. Fresh media was replaced bi-weekly.

None of the 65 transformed immature cotyledons cultured on M2 medium showed any green embryogenic callus formation (Table 3). In contrast, of the 127 transformed immature cotyledons cultured on S30 medium 17 showed green embryogenic callus formation resulting in a transformation frequency of 13.4%. See Table 3.

TABLE 3
The effect of glutamine and L-methionine (S30 medium) on
transformation frequency of soybean using chlorsulfuron selection

| | Number of immature | Number of green embryogenic |
|---|---|---|
| | cotyledons | callus and (% transformation |
| Medium | transformed | frequency) |
| M2 | 65 | 0 (0%) |
| S30 | 127 | 17 (13.4%) |

As shown in Tables 2 and 3 both hygromycin (Table 2) and chlorsulfuron (Table 3) selection produced stably transformed events with higher transformation frequencies in S30 medium when compared to the transformation frequencies obtained in M2 medium.

### Example 7. Effect of Immature Cotyledon Size and Glutamine Concentration in Soybean Elite Varieties 93Y53 and 93Y83

To determine the effect of immature cotyledon size on transformation efficiency, immature cotyledons 3 to 4 mm, 4 to 5 mm and 5 to 7 mm in length from elite varieties 93Y53 and 93Y83 were transformed with AGL-1 harboring the binary test plasmid B described in Example 2. Four days after co-cultivation in M5 medium, transformed immature cotyledons were cultured on S30 medium (M2 medium supplemented with 1 mM L-methionine and 30.6 mM glutamine) or GLU15 medium (M2 medium supplemented with 1 mM L-methionine and 15 mM glutamine) and each further supplemented with 300 mg/L timentin without a selection agent for recovery. After a 1-week recovery, immature cotyledons were cultured for 6 weeks on S30 medium or GLU15 medium supplemented with 300 mg/L timentin and 10 μg/L chlorsulfuron. Fresh media was replaced bi-weekly. The results shown in Table 4 demonstrate that immature cotyledons 3-4 mm in length of both elite varieties, 93Y53 and 93Y83, showed higher transformation efficiencies than larger (5-7 mm in length) immature cotyledons. Transformed immature cotyledons cultured on M2 medium did not show any green embryogenic callus (data not shown). Additional experiments used immature cotyledons in the range of 0.5 to 9.0 mm. These experiments also showed higher transformation frequencies when cultured on S30 medium (data not shown).

TABLE 4
Effect of immature cotyledon size and glutamine concentration
in soybean elite varieties 93Y53 and 93Y83 6 weeks after
transformation

| | | | | Number |
|---|---|---|---|---|
| | | | | of events |
| | | | Number | at callus |
| | | Immature | of | level and |
| | | cotyledon | immature | (% |
| | | length | cotyledons | transformation |
| Cultivar | Medium | (mm) | transformed | frequency) |
| Elite | S30 | 3-4 | 82 | 5 (6.1%) |
| 93Y53 | | 4-5 | 82 | 3 (3.6%) |
| | | 5-7 | 84 | 1 (1.2%) |
| | GLU15 | 3-4 | 42 | 2 (4.8%) |
| | | 4-5 | 42 | 6 (14.3%) |
| | | 5-7 | 42 | 0 (0%) |
| Elite | S30 | 3-4 | 127 | 17 (13.4%) |
| 93Y83 | | 4-5 | 40 | 2 (5%) |
| | | 5-7 | 40 | 0 (0%) |
| | GLU15 | 3-4 | 43 | 29 (67.5%) |
| | | 4-5 | 43 | 16 (37.2%) |
| | | 5-7 | 43 | 2 (4.6%) |

### Example 8. Agrobacterium Strains

Four Agrobacterium strains (AGL-1, EHA105, GV3101, and LBA4404) were compared for their ability to mediate transformation of soybean immature cotyledons. Immature cotyledons of elite variety 93Y21 were transformed with A. tumefaciens AGL-1, EHA105, GV3101, and LBA4404 harboring binary test plasmid B in M5 co-cultivation medium. Following 4 days of co-cultivation, T-DNA transfer was determined by observing TagRFP transient expression. All four strains produced high levels of transient TagRFP expression (data not shown). Of the four Agrobacterium strains (AGL-1, EHA105, GV3101, and LBA4404) tested, LBA4404 showed the least TagRFP expression (data not shown). No significant differences in transformed tissue viability were observed in the four strains (AGL-1, EHA105, GV3101, and LBA4404) tested (data not shown).

### Example 9. Effect on Transformation Frequency of Soybean Immature Cotyledon Pre-Incubation in S30 Medium

Testing to determine the effect on transformation frequency of pre-incubation of soybean immature cotyledons in S30 liquid medium was carried out using elite soybean cultivar 93Y21. Freshly isolated immature cotyledons of elite soybean variety 93Y21 were transformed with AGL-1 harboring the binary test plasmid B described in Example 2. At the same time 200-300 immature cotyledons were pre-cultured in a 250 mL sterile flask containing 50 mL S30 liquid medium at 25°-26° C. with an 18-hour photoperiod at 35-60 μE/m²/s light intensity on a rotary shaker at 100 rpm for 2 days. Following pre-incubation of soybean immature cotyledons in S30 liquid medium for 2 days, 20-25 immature cotyledons were transferred into a sterile glass tube (16×100 mm) with 400 μL inoculum of A. tumefaciens AGL-1 and transformation, using the same protocol with chlorsulfuron 10 μg/L selection, was carried out as described in Example 3.

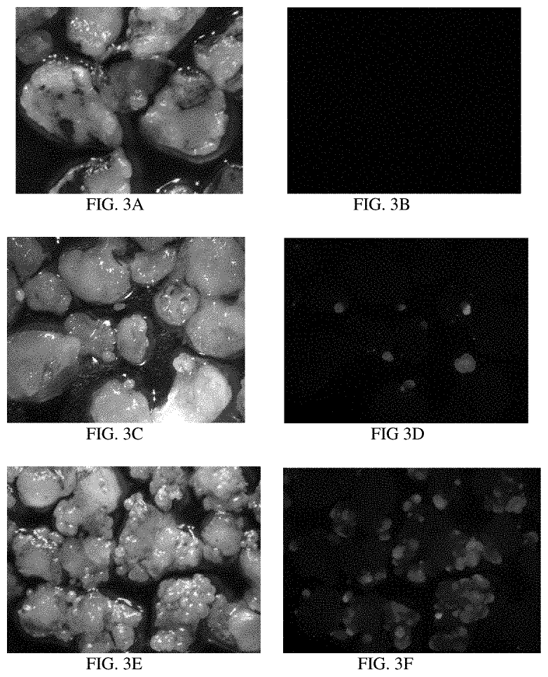

The results of this experiment are shown in Table 5 and FIG. 3. Transformation efficiencies after pre-culture of immature cotyledons in S30 medium for 2 days ranged from >114-303% at an embryogenic callus level. The pre-culture treatment in the S30 medium increased the transformation efficiencies significantly compared to non-pre-culture treatments (0 day). See Table 5.

TABLE 5
Effect of pre-culture in S30 medium on the rate of trans-
formation frequency of immature cotyledons.

| | Number of | Number of events |
|---|---|---|
| | immature | at callus level and |
| Days of | cotyledons | (% transformation |
| pre-culture | transformed | frequency) |
| 0 | 40 | 9 (22.5%) |
| | 46 | 5 (10.9%) |
| | 41 | 13 (31.7%) |
| 2 | 36 | 109 (303%) |
| | 43 | 49 (114%) |
| | 47 | 72 (153%) |

### Example 10. Molecular Analyses

Quantitative polymerase chain reaction (qPCR) for putative T0 events of soybean elite cultivar 93Y21 transformed with AGL-1 harboring the binary test plasmid B (an intron-containing the Tag red fluorescent protein (TagRFP) gene driven by the Arabidopsis UBQ10 promoter and an intron-containing the GM-HRA gene driven by the GM SAMS promoter) was performed using primers for the GM-SAMS promoter in the ALS expression cassette (GM-HRA) and the GM-UBQ3 terminator in TagRFP expression cassette. qPCR reactions were performed in accordance with the manufacturer's protocols provided by Applied Biosystems (Life Technologies Corporation, Carlsbad, Calif., USA) for real-time qPCR machines 7500 and 7900HT. A total of 235 events were analyzed. The probability of qPCR positive events containing at least one copy of both genes (TagRFP and GM-HRA) was 70.2% (Table 6). The transformation frequency of qPCR single copy events containing both genes was 20.4%. The percentage of escapes was 29.8%. The percentage of escapes was significantly reduced when the rooting medium was supplemented with 5 μg/L chlorsulfuron (data not shown).

TABLE 6
qPCR for putative events of elite soybean cultivar 93Y21
transformed with A. tumefaciens AGL-1 harboring
the binary test plasmid B after selection
with 10 μg/L chlorsulfuron

| | # Positive | | | |
|---|---|---|---|---|
| | of Events | | # of Single | # of Events with |
| | and (% | # of Escapes | Copy Events | more than 1 |
| | trans- | and (% | and (% | Copy and |
| Total T0 | formation | transformation | transformation | (% transformation |
| events | frequency) | frequency) | frequency) | frequency) |
| 235 | 165 | 70 | 48 | 117 |
| | (70.2%) | (29.8%) | (20.4%) | (49.8%) |

### Example 11. Fertile Plant Regeneration

Stably transformed soybean plants from elite cultivars showing normal phenotypes were regenerated, successfully transplanted to soil, and grown in the greenhouse (data not shown). The transformed plants were fertile with normal phenotypes and T1 progeny were recovered (data not shown). The present method facilitates the production of transgenic plants within about 4 months from the initiation of transformation to transferring plantlets to soil for growth in the greenhouse.

### Example 12. Use of Modified S30 Medium to Generate Target Tissue for Biolistic-Mediated Soybean Transformation

The methods described in this Example 12 can be used to transform any soybean variety. Immature pods were collected from soybean plants and opened to retrieve immature seeds of about 2-8 mm in length. Immature seeds were collected and surface sterilized as described in Example 3. Surface sterilized seeds were cut open under a microscope or under magnification. Typically, each immature seed's embryonic axis was cut off, and the two cotyledon pieces were released. Immature cotyledons were collected and transferred to flasks containing liquid Modified S30 medium. Immature cotyledons of about 0.5 to 9.0 mm in length were used. However, other sizes can also be used to generate target tissue for biolistic-mediated transformation purposes. Fresh Modified S30 medium was replaced weekly. The concentration of 2,4-D in the Modified S30 medium to generate embryogenic target tissue for biolistic-mediated transformation may be from about 11 mg/L to about 18 mg/L, and preferably was about 17 mg/L.

Highly regenerable embryogenic calli were induced using Modified liquid S30 medium. This induction was more rapid than that reported in the literature (data not shown). Thirty-two (32) batches of immature cotyledons were isolated from immature seeds, and 6-59 immature cotyledons per flask were cultured in Modified S30 medium to induce embryogenic callus. At the end of a 4-week culture in Modified S30 medium, more than 77% of the immature cotyledons produced embryogenic callus, and more than 57% of the immature cotyledons had greater than 50% of their surface covered with embryogenic calli. Conventional methods using solid medium to initiate embryogenic callus is typically about 3-fold less efficient and takes at least twice as long (data not shown) as the method described in this Example 12.

Once embryogenic calli were initiated in Modified S30 medium, they were maintained in M2 medium (Table 1) for subsequent biolistic-transformations. For elite cultivar 93Y21, highly proliferative early stage embryogenic callus clusters were selected and transferred into M2 medium after 3-4 weeks of induction in Modified S30 medium. For soybean genotypes such as Jack, which tends to have quicker response, highly proliferative early stage embryogenic callus clusters were selected and transferred into M2 medium after 2-3 weeks of induction in Modified S30 medium. After 1-2 weeks of culture in M2 medium, the established green, embryogenic calli were ready for biolistic-mediated transformation. Embryogenic callus has been maintained in M2 medium for up to 1 year, preferably less than 3 months prior to biolistic transformation. Based on these results, one skilled in the art is able to determine culture conditions for other soybean genotypes.

### Example 13. Agrobacterium-Mediated Immature Cotyledon Transformation Using the Bar Gene as a Selectable Marker

The bar (bialaphos resistance) gene was used as a selectable marker to obtain transgenic events using the methods described herein. Bialaphos concentrations of 10 mg/L and 30 mg/L were used in this Example 13. Two co-cultivation temperature treatments of 21° C. and 25° C. were also included. Four plates were included as replicates for each treatment. and 30 immature cotyledons were infected per plate for a total of 120 immature cotyledons for each bialaphos and temperature combination. Immature cotyledons were cut, pre-cultured, and infected as described in other examples. The transformation vector contained the synthetic SCP promoter driving the bar gene and the Arabidopsis UBQ10 promoter driving an insect resistance gene. Agrobacterium strain AGL-1 harboring the construct at an optical density (OD) of 0.5 was used for transformation. Infected explants were co-cultivated with Agrobacterium for 5 days. Molecular analysis identified transgenic events containing the bar gene and the insect resistance gene (data not shown).

### Example 14. Agrobacterium-Mediated Immature Cotyledon Transformation for Gene Editing

Immature cotyledons were cut, pre-cultured, and infected as described in other examples. Two constructs were used for gene editing purposes: one targeting two FAD2 genes (1A and 1B) and another targeting two FAD3 genes (3a and 3b). Each construct contained a guide RNA, Cas9 and herbicide resistance (selectable marker) gene components. Agrobacterium strain AGL-1 harboring each construct at an optical density (OD) of about 0.5 was used for transformation. Infected explants were co-cultivated with Agrobacterium for 4 days. After recovery and selection, events were collected for embryo maturation and development. Leaf disc samples were used for qPCR analysis. The results are summarized in Table 7.

TABLE 7
Agrobacterium-mediated immature cotyledon transformation
gene editing results.

| | No. of events | No. of events |
|---|---|---|
| | with Cas9 and | with edits in |
| Target Genes | selectable marker | all target genes |
| FAD2 genes (1A and 1B) | 3 | 3 |
| FAD2 genes (1A and 1B) and | 4 | 4 |
| FAD3 genes (3a and 3b) | | |

### Example 15. Effect of Casein Hydrolysate to Improve Embryo Maturation

Typically, prior to germination, SHaM maturation medium (Soybean Histodifferentiation and Maturation Medium, Schmidt, M. A., D. M. Tucker, E. B. Cahoon, and W. A. Parrott. 2005. Towards normalization of soybean somatic embryo maturation Plant Cell Rep. 24:383-391) is used to mature embryos of model soybean genotypes such as Jack or Williams. This Example 15 demonstrated that improved regeneration was achieved for elite lines using SHaM medium supplemented with casein hydrolysate. The effect of higher concentrations of glutamine (50%, and 100% more than the glutamine concentration in SHaM medium) and the addition of 1 g/L and 2 g/L of casein hydrolysate to SHaM medium was examined. A liquid medium format was used. Five (5) treatments were used as shown in Table 8. Each treatment had eight plates as replicates, and there were 48 samples for each treatment. Small embryogenic calli (about 1 mm in size) induced from elite soybean 93Y21 were used. Each embryogenic callus was placed in a well of a 6-well culture plate with 6 ml of liquid medium according to the treatments described in Table 8. After 15 days of culture, the callus pieces became clusters of embryos. The embryo clusters were quickly blotted on sterile filter paper and weighed, and embryo quality was evaluated as follows: 1—tight embryo cluster with no elongation; 2—tight embryo cluster with some elongation; 3—embryos elongated but no defined cotyledons and no separation; and 4—elongated embryos with defined cotyledons and good separation. Embryos were then dried for up to about 17 days and were transferred to M8 germination medium (see Table 1). After about 21 days of culture on M8 germination medium shoots taller than 1 cm were counted. SHaM medium supplemented with 1 g/L casein hydrolysate gave the best combined score of embryo quality, germination frequency and elongated shoots. See Table 8. This medium has been used with similar results in both liquid and solid form for embryo maturation in soybean transformations using both biolistic-mediated and bacteria-mediated transformation methods.

TABLE 8
Addition of casein hydrolysate in maturation medium improves embryo
quality and germination for elite soybean lines

| | | | | Avg. Percentage | Avg. No. of |
|---|---|---|---|---|---|
| | | | Mean | of Samples with | Regenerated |
| | No. | Weight | Embryo | Shoots (based on | Shoots |
| Medium | Samples | (gram) | Score | plates) | (>1 cm)/sample |
| SHaM | 48 | 0.52 | 2.24 | 79.13 | 3.4 |
| SHaM + 50% more | 48 | 0.43 | 2.51 | 83.37 | 3.1 |
| Glutamine | | | | | |
| SHaM + 100% more | 48 | 0.39 | 2.55 | 68.87 | 3.3 |
| Glutamine | | | | | |
| SHaM + 1 g/L Casein | 48 | 0.50 | 2.77 | 81.25 | 4.0 |
| Hydrolysate | | | | | |
| SHaM + 2 g/L Casein | 48 | 0.33 | 2.83 | 68.87 | 4.2 |
| Hydrolysate | | | | | |

### Example 16. Immature Cotyledons Pre-Cultured in Modified S30 Medium Used for Direct Biolistic-Mediated Transformation

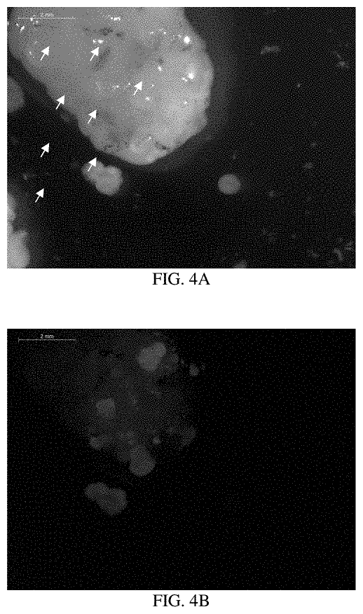

Immature cotyledons were pre-cultured in Modified S30 medium and targeted for direct biolistic-mediated DNA transformation. Sixty (60) immature cotyledons were isolated from immature seeds as described in previous examples. These immature cotyledons were pre-cultured for 10 days in liquid Modified S30 medium. After 10 days of pre-culture, twenty (20) immature cotyledons were placed on the surface of M2 solid medium supplemented with 40 mg/L of 2,4-D in the center of small petri plates (60×15 mm) for bombardment. A plasmid DNA mixture containing 2 constructs: one construct containing YFP florescence marker (GM UBQ::ZS-YELLOW) and a hygromycin selectable marker gene (35S::HYG)) and a second construct containing GM LTP3::AtWUS (PCT Publication No. WO 2017/112006) were used for the bombardment. The immature cotyledons were bombarded with the plasmid DNA mixture at a concentration of 9 picogram/basepair/shot. After bombardment, the bombarded immature cotyledons were transferred to liquid Modified S30 medium. After 4 weeks of selection, 16 hygromycin resistant calli emerged from the surface of explants (FIG. 4A and FIG. 4B). Thirteen transgenic events passed through selection and embryo maturation and were placed on M8 germination medium.

### Example 17. Immature Cotyledons Pre-Cultured in S30 Medium Used for Direct Biolistic-Mediated Transformation

Immature cotyledons were pre-cultured in S30 medium and targeted for direct biolistic-mediated DNA transformation. Immature cotyledons 2.5-4 mm in size were isolated from immature seeds as described in Example 3. These immature cotyledons were pre-cultured for 2 days in liquid S30 medium. After 2 days of pre-culture, 35-223 immature cotyledons were placed onto a sterile filter paper on the surface of S30 solid medium or M5 solid medium solidified with 5 g/L TC agar in the center of petri plates (100×15 mm) for bombardment. A plasmid (Plasmid C) containing an ALS selectable marker and a fluorescent protein (DsRed) was used in this Example 17. The immature cotyledons were bombarded with Plasmid C DNA at a concentration of 30 picogram/base pair/shot. After bombardment, the bombarded immature cotyledons were co-cultivated onto a sterile filter paper on the surface of S30 solid medium or M5 solid medium. After 1-2 days of co-cultivation, immature cotyledons were then transferred to a 250 mL sterile glass flask containing 40-50 mL S30 medium without selection, and were cultured at 25-26° C. with an 18-hour photoperiod at 35-60 μE/m²/s light intensity for 7 days on rotary shaker at 100 rpm for a recovery period.

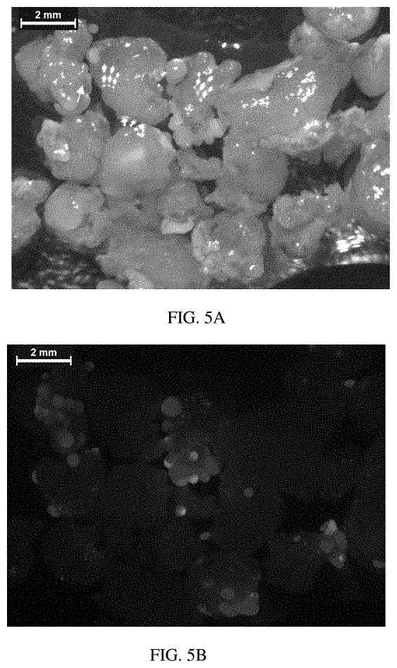

Following the recovery period, S30 medium containing a selection agent (10 μg/L chlorsulfuron) was used for the selection of stable transformants. The S30 supplemented with 10 μg/L chlorsulfuron was replaced bi-weekly and cultured at 25-26° C. with 18-hour photoperiod at 35-60 μE/m²/s light intensity on a rotary shaker at 100 rpm. After 4-6 weeks on S30 supplemented with 10 μg/L chlorsulfuron, transformed tissue became visible as green tissue and DsRed positive under a fluorescent microscope (FIG. 5A and FIG. 5B) against a background of bleached, less healthy tissue. After 6 weeks of selection at a concentration of 30 picogram/base pair/shot, >267 putative events (16.6% transformation efficiency) showing DsRed positive expression in most of the chlorsulfuron resistant green embryogenic callus from 1,610 immature cotyledons were observed as shown in Table 9.

After 1-4 days of co-cultivation on M5 solid medium, immature cotyledons were then transferred to a 250 mL sterile glass flask containing 40-50 mL S30 medium without selection, and were cultured at 25-26° C. with an 18-hour photoperiod at 35-60 μE/m²/s light intensity for 7 days on rotary shaker at 100 rpm for a recovery period. The immature cotyledons were then transferred to S30 medium with selection. After 6 weeks of selection at a concentration of 3 picogram/base pair/shot, >197 putative events (35.8% transformation efficiency) showing DsRed positive expression in most of the chlorsulfuron resistant green embryogenic calli from 551 immature cotyledons were observed as shown in Table 10.

TABLE 9
Transformation efficiencies bombarded at a concentration of
30 picogram/base pair/shot

| | Co- | Co- | | Putative | % |
|---|---|---|---|---|---|
| Shot | cultivation | cultivation | Explants | events | transformation |
| # | medium | days | # | # | efficiency |
| 1 | S30 | 1 | 211 | 6 | 2.8 |
| 2 | M5 | 1 | 35 | 17 | 48.6 |
| 3 | S30 | 1 | 123 | 19 | 15.5 |
| 4 | M5 | 1 | 207 | 94 | 45.4 |
| 5 | M5 | 2 | 188 | 44 | 23.4 |
| 6 | S30 | 2 | 129 | 12 | 9.3 |
| 7 | S30 | 2 | 184 | 14 | 7.6 |
| 8 | S30 | 2 | 188 | 8 | 4.3 |
| 9 | M5 | 2 | 122 | 11 | 9.0 |
| 10 | M5 | 2 | 223 | 42 | 18.8 |
| | | | 1,610 | 267 | 16.6 |

TABLE 10
Transformation efficiencies bombarded at a concentration of
3 picogram/base pair/shot

| | Co- | Co- | | Putative | % |
|---|---|---|---|---|---|
| Shot | cultivation | cultivation | Explants | events | transformation |
| # | medium | days | # | # | efficiency |
| 1 | M5 | 1 | 127 | 51 | 40.2 |
| 2 | M5 | 2 | 141 | 16 | 11.6 |
| 3 | M5 | 3 | 145 | 72 | 49.7 |
| 4 | M5 | 4 | 138 | 58 | 42.00 |
| | | | 551 | 197 | 35.8 |

### Example 18. Immature Cotyledons Used for Direct Biolistic-Mediated Transformation

Immature cotyledons 4-7 mm in size were targeted for direct biolistic-mediated DNA delivery. These immature cotyledons were isolated as in Example 3 and were directly bombarded with plasmid DNA (Plasmid D) containing a hygromycin selectable marker and a fluorescent marker (ZsYellow). 12-18 immature cotyledons were placed in the center of the plates and were bombarded with the Plasmid D DNA at 9 picogram/base pair/shot for once or twice. After bombardment, the bombarded immature cotyledons were cultured in about 50 ml liquid S30 medium in 250 ml flasks without selection for one week (25-26° C. with an 18-hour photoperiod at 35-60 μE/m²/s light intensity for 7 days on rotary shaker at 100 rpm), and then transferred to liquid S30 medium with 25 mg/L of hygromycin for selection. The selection medium was replaced bi-weekly. The flasks were cultured at 25-26° C. with an 18-hour photoperiod at 35-60 μE/m²/s light intensity on rotary shaker at 100 rpm.

After 4-6 weeks on selection medium (S30 medium supplemented with 25 mg/L of hygromycin), transformed tissue became visible as green tissue and ZsYellow positive under the fluorescent microscope. After 6 weeks of selection, events showing ZsYellow fluorescence were moved to maturation medium (M7). 13 ZsYellow positive events were obtained from 80 immature cotyledons bombarded once (16%), and 12 ZsYellow positive events were obtained from 78 immature cotyledons twice (15%). The data is summarized in Table 11.

TABLE 11
Transformation efficiencies bombarded at a concentration of
9 picogram/base pair/shot

| | | | # | | |
|---|---|---|---|---|---|
| | | | Hgy | # | |
| # | IC Size | # | resistant | YFP + | YFP + |
| Shots | (mm) | IC | Events | Events | Frequency |
| 1x | 4.9 | 18 | 7 | 5 | 27.70% |
| 1x | 5.4 | 16 | 1 | 1 | 6.50% |
| 1x | 5.7 | 12 | 0 | 0 | 0 |
| 1x | 5 | 16 | 8 | 5 | 31% |
| 1x | 4.8 | 18 | 4 | 2 | 11.10% |
| 2X | 5.7 | 16 | 0 | 0 | 0 |
| 2X | 5.2 | 14 | 5 | 5 | 35.70% |
| 2X | 6.2 | 14 | 0 | 0 | 0 |
| 2X | 5.1 | 16 | 7 | 5 | 31.20% |
| 2X | 5.4 | 18 | 3 | 2 | 11.10% |

### Example 19. Use of Immature Cotyledons for Production of Site Specific Integration (SSI) Target Lines

Immature cotyledons of elite soybean lines are cut, pre-cultured, and infected as described in other examples. In the alternative, no per-culture step is used prior to infection. The immature cotyledons of the elite soybean lines are infected with a bacterial strain disclosed herein, comprising a polynucleotide comprising at least one recombination site recognized by a recombinase to generate a target site in the genome of the elite soybean line. Target sites include both native and non-native sites. The target site may comprise at least 1, 2, 3, 4, 5, 6 or more recombination sites for site-specific recombination. The target locus may comprise two identical or two non-identical recombination sites. One or more intervening sequences may be present between the recombination sites of the target locus. Intervening sequences include but are not limited to native genomic sequence, linkers, adapters, selectable markers, polynucleotides of interest, other recombination sites, promoters and/or other sites that aid in vector construction or analysis. Various polynucleotides of interest may be employed between the recombination sites of the target site. In addition, the recombination sites of the target site can be located in various positions, including, for example, within intron sequences, coding sequences, or untranslated regions. The recombination sites employed can be recombinogenic or identical sites, or dissimilar or non-identical sites. Recombinogenic or identical sites have the same or similar sequence, and in the presence of the appropriate recombinase, will recombine with one another. Dissimilar or non-identical recombination sites have distinct sequences (i.e., have at least one nucleotide difference), and in the presence of the appropriate recombinase, will have minimal or no recombination with each other. Each recombination site within the set of nonidentical recombination sites is biologically active and therefore can recombine with an identical site.

Plants containing the target site are regenerated as described in other examples. It is expected that large numbers of transgenic soybean target lines will be generated using immature cotyledons. The target lines so generated will be clean single copy (=matched border) transgenic soybean target lines having normal phenotypes and yields in field testing. These SSI target lines can be used for retransformation using any transformation system.

All patents, publications and patent applications mentioned in the specification are indicative of the level of those skilled in the art to which this disclosure pertains. All patents, publications and patent applications are herein incorporated by reference in its entirety to the same extent as if each individual patent, publication or patent application was specifically and individually indicated to be incorporated by reference in its entirety.

Although the foregoing disclosure has been described in some detail by way of illustration and example for purposes of clarity of understanding, certain changes and modifications may be practiced within the scope of the appended claims.

That which is claimed:

1. A method of transforming a dicot immature cotyledon to produce a fertile transgenic dicot plant, comprising:
a) infecting and co-cultivating the immature cotyledon with bacteria containing a plasmid in M5 medium;
b) recovering transformed immature cotyledons in S30 medium;
c) selecting the transformed immature cotyledon in S30 medium further comprising a selection agent;
d) obtaining transformed embryogenic callus from the immature cotyledon; and
e) regenerating transformed plants from the embryogenic callus.

2. The method of claim 1, wherein the immature cotyledon and the bacteria are co-cultivated on filter paper in a).

3. The method of claim 1, wherein the infecting is performed by wounding the immature cotyledon.

4. The method of claim 1, wherein the immature cotyledon of a) is about 0.5-9.0 mm in length.

5. The method of claim 1, wherein the bacteria of a) is a disarmed Agrobacteria, an Ochrobactrum bacteria or a Rhizobiaceae bacteria.

6. The method of claim 1, wherein the plasmid comprises a gene of interest and/or a selectable marker.

7. The method of claim 1, wherein the plasmid comprises a guide RNA, a target nucleotide present in the dicot immature cotyledon, and a Cas endonuclease, wherein the guide RNA and the Cas endonuclease are capable of forming a complex that enables the Cas endonuclease to introduce a double strand break at a target site in the genome of the dicot immature cotyledon, wherein at least one nucleotide of the target nucleotide is modified.

8. The method of claim 1, wherein the plasmid comprises a promoter operably linked to a nucleotide sequence encoding an engineered meganuclease wherein the engineered meganuclease is capable of specifically binding to and creating a double strand break in a target sequence present in the dicot immature cotyledon and wherein the promoter is capable of driving expression of an operably linked nucleotide sequence in the dicot immature cotyledon, wherein the nucleotide sequence comprises an alteration in the genome of the dicot immature cotyledon.

9. The method of claim 1, wherein the selection agent of c) is an antibiotic, an herbicide, or a positive selection agent.

10. The method of claim 9, wherein the selection agent is hygromycin, spectinomycin, kanamycin, a sulfonylurea herbicide, an imidazolinone herbicide, bialaphos, phosphinothricin, glyphosate, or a combination thereof.

11. The method of claim 10, wherein the sulfonylurea herbicide is chlorsulfuron, ethametsulfuron, or a combination thereof.

12. The method of claim 10, wherein the imidazolinone herbicide is imazapyr.

13. The method of claim 1, wherein the dicot plant is selected from the group consisting of kale, cauliflower, broccoli, mustard plant, cabbage, pea, clover, alfalfa, broad bean, tomato, cassava, soybean, peanut, canola, alfalfa, sunflower, safflower, tobacco, Arabidopsis, and cotton.

14. The method of claim 1, wherein the dicot plant is soybean.

15. A method of transforming a dicot immature cotyledon to produce a fertile transgenic dicot plant, comprising:
a) bombarding the immature cotyledon with a DNA or a ribonucleoprotein;
b) selecting the transformed immature cotyledon in S30 medium further comprising a selection agent;
c) obtaining transformed embryogenic callus from the immature cotyledon; and
d) regenerating transformed plants from the embryogenic callus.

16. The method of claim 15, wherein the immature cotyledon of a) is about 0.5-9.0 mm in length.

17. The method of claim 15, wherein the DNA or a ribonucleoprotein comprises a gene of interest and/or a selectable marker.

18. The method of claim 15, wherein the DNA or the ribonucleoprotein comprises a guide RNA, a target nucleotide present in the dicot immature cotyledon, and a Cas endonuclease, wherein the guide RNA and the Cas endonuclease are capable of forming a complex that enables the Cas endonuclease to introduce a double strand break at a target site in the genome of the dicot immature cotyledon, wherein at least one nucleotide of the target nucleotide is modified.

19. The method of claim 15, wherein the DNA or the ribonucleoprotein comprises a promoter operably linked to a nucleotide sequence encoding an engineered meganuclease wherein the engineered meganuclease is capable of specifically binding to and creating a double strand break in a target sequence present in the dicot immature cotyledon and wherein the promoter is capable of driving expression of an operably linked nucleotide sequence in the dicot immature cotyledon, wherein the nucleotide sequence comprises an alteration in the genome of the dicot immature cotyledon.

20. The method of claim 15, wherein the selection agent of b) is an antibiotic, an herbicide, or a positive selection agent.

21. The method of claim 20, wherein the selection agent is hygromycin, spectinomycin, kanamycin, a sulfonylurea herbicide, an imidazolinone herbicide, bialaphos, phosphinothricin, glyphosate, or a combination thereof.

22. The method of claim 21, wherein the sulfonylurea herbicide is chlorsulfuron, ethametsulfuron, or a combination thereof.

23. The method of claim 21, wherein the imidazolinone herbicide is imazapyr.

24. The method of claim 15, wherein the dicot plant is selected from the group consisting of kale, cauliflower, broccoli, mustard plant, cabbage, pea, clover, alfalfa, broad bean, tomato, cassava, soybean, peanut, canola, alfalfa, sunflower, safflower, tobacco, Arabidopsis, and cotton.

25. The method of claim 15, wherein the dicot is soybean.

26. A method of improving bacteria-mediated transformation frequency of a dicot immature cotyledon, comprising:
culturing the immature cotyledon for a period of from about one day to about ten days in S30 medium prior to bacteria-mediated transformation to provide a pre-cultured immature cotyledon, wherein the bacteria-mediated transformation frequency of the pre-cultured immature cotyledon is improved when compared to the bacteria-mediated transformation frequency of a non-pre-cultured immature cotyledon.

27. A method of improving biolistic-mediated transformation frequency of a dicot immature cotyledon, comprising:
culturing the immature cotyledon for a period of from about one day to about ten days in S30 medium prior to biolistic-mediated transformation to provide a pre-cultured immature cotyledon, wherein the biolistic-mediated transformation frequency of the pre-cultured immature cotyledon is improved when compared to the biolistic-mediated transformation frequency of a non-pre-cultured immature cotyledon.

28. A method of generating embryogenic callus for biolistic-mediated transformation, comprising:
culturing an immature cotyledon for a period of from about ten days to about five weeks in S30 medium to provide the embryogenic callus for biolistic-mediated transformation.
